(19) United States
(12) Patent Application Publication
(10) Pub. No.: US 2021/0052193 A1
(43) Pub. Date: Feb. 25, 2021

(54) ELECTRONIC DEVICE, DISPLAY METHOD, AND DISPLAY SYSTEM

(71) Applicant: KYOCERA Corporation, Kyoto (JP)

(72) Inventor: Hiromi AJIMA, Kawasaki-shi, Kanagawa (JP)

(73) Assignee: KYOCERA Corporation, Kyoto (JP)

(21) Appl. No.: 16/606,666

(22) PCT Filed: Apr. 10, 2018
(86) PCT No.: PCT/JP2018/015129

§ 371 Date: Oct. 18, 2019

(30) Foreign Application Priority Data
Apr. 25, 2017 (JP) 2017-086608

(51) Int. Cl. A61B 5/107; A61B 5/00

(52) U.S. Cl. CPC A61B 5/1077; A61B 5/7425; A61B 5/7475; A61B 5/1079; A61B 5/4866; G01B 3/1069; A61B 5/4848; A61B 5/6898; A61B 2562/0219; A61B 2560/0475; A61B 5/0022; A61B 5/4806

(57) ABSTRACT

An electronic device includes a measurement unit that measures a contour of an abdomen and a controller that displays the contour on a display. The controller displays a first contour and a second contour that are measured at different times in overlap on the display.

### CROSS-REFERENCE TO RELATED APPLICATION

The present application claims priority to and the benefit of Japanese Patent Application No. 2017-086608 filed Apr. 25, 2017, the entire contents of which are incorporated herein by reference.

### TECHNICAL FIELD

The present disclosure relates to an electronic device, a display method, and a display system.

### BACKGROUND

Computed tomography (CT) is a known method for measuring the visceral fat area of an abdominal cross-section. A method for visually displaying the visceral fat area measured by CT is also known. For example, patent literature (PTL) 1 discloses an apparatus for displaying the fat area using a circle.

### CITATION LIST

### Patent Literature

PTL 1: JP2002-191563A

### SUMMARY

An electronic device according to an embodiment includes a measurement unit configured to measure a contour of an abdomen and a controller configured to display the contour on a display. The controller is configured to display a first contour and a second contour that are measured at different times in overlap on the display.

A display method according to an embodiment is a display method to be executed by an electronic device. The display method includes measuring a contour of an abdomen and displaying a first contour and a second contour that are measured at different times in overlap on a display.

A display system according to an embodiment includes a measurement unit configured to measure a contour of an abdomen and a controller configured to display the contour on a display. The controller is configured to display a first contour and a second contour that are measured at different times in overlap on the display.

### BRIEF DESCRIPTION OF THE DRAWINGS

In the accompanying drawings:

FIG. 1 is a schematic perspective view illustrating the appearance of a smartphone according to a first embodiment;

FIG. 2 is a schematic front view illustrating the appearance of the smartphone according to the first embodiment;

FIG. 3 is a schematic back view illustrating the appearance of the smartphone according to the first embodiment;

FIG. 4 is a schematic block diagram illustrating the functions of the smartphone according to the first embodiment;

FIG. 5 is a schematic diagram illustrating measurement of the contour of an abdominal cross-section according to the first embodiment;

FIG. 6 is a flowchart for measuring the contour of a cross-section according to the first embodiment;

FIGS. 7A and 7B illustrate an example of orientation and movement amount according to the first embodiment;

FIG. 8 is an example record of orientation information and movement information according to the first embodiment;

FIG. 9 illustrates the contour of a cross-section calculated in the first embodiment;

FIG. 10 illustrates correction using an actual measured value according to the first embodiment;

FIG. 11 schematically illustrates an electronic tape measure according to the first embodiment;

FIG. 12 illustrates example data stored on a storage of the smartphone according to the first embodiment;

FIG. 13 is a flowchart for deriving a visceral fat area estimation formula and a subcutaneous fat area estimation formula;

FIGS. 14A, 14B, and 14C are schematic diagrams illustrating example classifications of the contour of the abdominal cross-section in the first embodiment;

FIG. 15 illustrates an example display by the smartphone according to the first embodiment;

FIG. 16 illustrates an example display by the smartphone according to the first embodiment;

FIG. 17 is a flowchart of the entire processing by the smartphone according to the first embodiment;

FIG. 18 is a schematic block diagram illustrating the functions of a smartphone according to a second embodiment;

FIG. 19 is a flowchart for measuring the contour of a cross-section according to the second embodiment;

FIG. 20 is an example record of orientation information and movement information according to the second embodiment;

FIG. 21 is a flowchart illustrating an example of processing up to display of the contour of an abdominal cross-section according to a third embodiment;

FIG. 22 illustrates an example orientation of a smartphone according to the third embodiment;

FIG. 23 is an example record formed by acquired information according to the third embodiment;

FIG. 24 illustrates the contour of a cross-section calculated and corrected in the third embodiment;

FIG. 25 illustrates an example display by the smartphone according to the third embodiment; and

FIG. 26 conceptually illustrates a device and a system according to an embodiment, the device including a communication interface.

### DETAILED DESCRIPTION

Since the apparatus disclosed in PTL 1 displays the measured visceral fat area, it is difficult for the user (subject) to understand the change over time in the abdominal cross-section. The present disclosure aims to provide an electronic device, a display method, and a display system that allow the user easily to understand the change over time in the abdominal cross-section.

Embodiments are described in detail with reference to the drawings.

In the present embodiment, a smartphone 1 is adopted as an example embodiment of an electronic device, and the case of measuring a human abdomen as an example of an object is described. The electronic device is not limited to the smartphone 1, nor is the object limited to a human abdomen. The object may be the abdomen of an animal.

### First Embodiment

The smartphone 1 is an electronic device that includes a first sensor that obtains orientation information, a device that obtains movement information, and a controller 10 that calculates the contour of a cross-section of an object. In the present embodiment, the device that obtains movement information includes a second sensor.

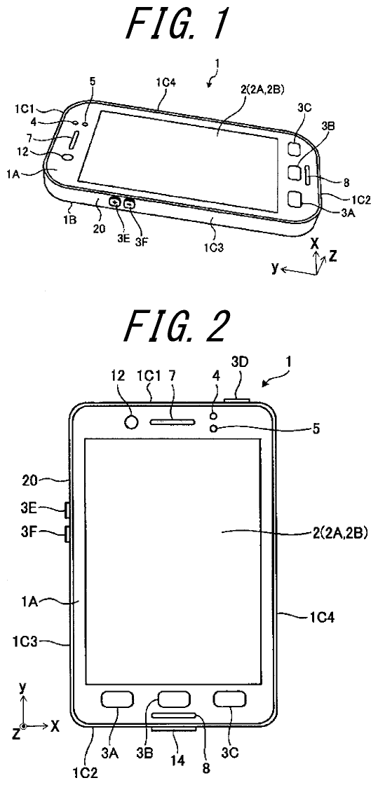

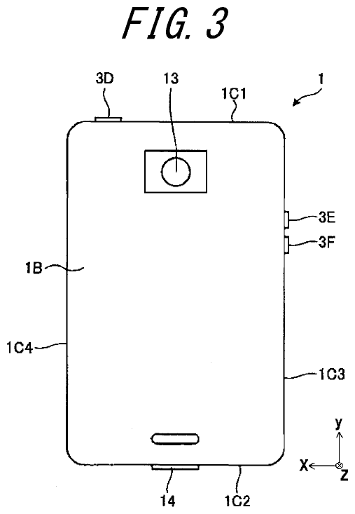

The appearance of the smartphone 1 according to the first embodiment is described with reference to FIGS. 1 to 3.

A housing 20 includes a front face 1A, a back face 1B, and side faces 1C1 to 1C4. The front face 1A is the front surface of the housing 20. The back face 1B is the back surface of the housing 20. The side faces 1C1 to 1C4 are side surfaces that connect the front face 1A and the back face 1B. The side faces 1C1 to 1C4 may be collectively referred to below as the side faces 1C without further distinction.

On the front face 1A, the smartphone 1 includes a touchscreen display 2, buttons 3A to 3C, an illuminance sensor 4, a proximity sensor 5, a receiver 7, a microphone 8, and a camera 12. The smartphone 1 includes a camera 13 on the back face 1B. The smartphone 1 also includes buttons 3D to 3F and a connector 14 on the side faces 1C. The buttons 3A to 3F may be collectively referred to below as the buttons 3 without further distinction.

The touchscreen display 2 includes a display 2A and a touchscreen 2B. The display 2A is provided with a display device such as a liquid crystal display, an organic electro-luminescence panel, or an inorganic electro-luminescence panel. The display 2A functions as a display for displaying characters, images, symbols, graphics, and the like.

The touchscreen 2B detects contact on the touchscreen 2B by a finger, stylus pen, or other such object. The touchscreen 2B can detect the position at which a plurality of fingers, stylus pens, or other objects contact the touchscreen 2B.

Any detection system may be used in the touchscreen 2B, such as a capacitive system, a resistive film system, a surface acoustic wave system (or an ultrasonic wave system), an infrared system, an electromagnetic induction system, or a load detection system. In a capacitive system, contact and proximity of an object such as a finger or stylus pen can be detected.

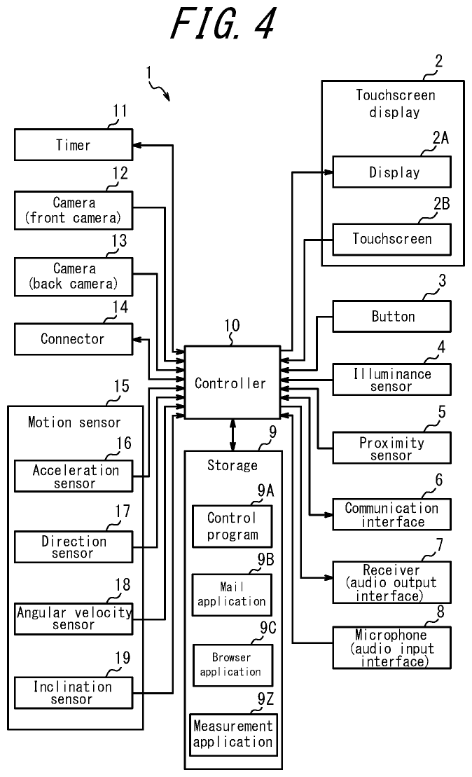

FIG. 4 is a block diagram illustrating the configuration of the smartphone 1. The smartphone 1 includes the touchscreen display 2, buttons 3, the illuminance sensor 4, a proximity sensor 5, a communication interface 6, the receiver 7, the microphone 8, a storage 9, the controller 10, a timer 11, the cameras 12 and 13, the connector 14, and a motion sensor 15.

As described above, the touchscreen display 2 includes a display 2A and a touchscreen 2B. The display 2A displays characters, images, symbols, graphics, and the like. The touchscreen 2B receives input of contact on a receiving area. In other words, the touchscreen 2B detects contact. The controller 10 detects a gesture on the smartphone 1. The controller 10 works together with the touchscreen 2B to detect an operation (gesture) on the touchscreen 2B (touchscreen display 2). The controller 10 also works together with the touchscreen 2B to detect an operation (gesture) on the display 2A (touchscreen display 2).

The buttons 3 are operated by the user. The buttons 3 include button 3A to button 3F. The controller 10 works together with the buttons 3 to detect an operation on the buttons. Examples of operations on the buttons include a click, a double-click, a push, a long push, and a multi-push.

For example, the buttons 3A to 3C may be a home button, a back button, or a menu button. In the present embodiment, touch-sensor buttons are used as the buttons 3A to 3C. The button 3D may, for example, be a power button for the smartphone 1. The button 3D may also function as a button to engage/release a sleep mode. The buttons 3E and 3F may, for example, be volume buttons.

The illuminance sensor 4 detects illuminance. The illuminance may, for example, be the intensity of light, brightness, or luminance. The illuminance sensor 4 may, for example, be used to adjust the luminance of the display 2A.

The proximity sensor 5 detects the presence of a nearby object without contact. The proximity sensor 5 may, for example, detect that the touchscreen display 2 has been brought close to a face.

The communication interface 6 communicates wirelessly. The communication method of the communication interface 6 is prescribed by a wireless communication standard. For example, a cellular phone communication standard such as 2G, 3G, or 4G may be used as the wireless communication standard. Examples of cellular phone communication standards include Long Term Evolution (LTE), W-CDMA, CDMA2000, PDC, Global System for Mobile communications (GSM® (GSM is a registered trademark in Japan, other countries, or both)), and Personal Handy-phone System (PHS). Examples of wireless communication standards include Worldwide Interoperability for Microwave Access (WiMAX), IEEE802.11, Bluetooth® (Bluetooth is a registered trademark in Japan, other countries, or both), IrDA, and NFC. The communication interface 6 may support one or more of the aforementioned communication standards.

The receiver 7 outputs an audio signal, transmitted from the controller 10, as sound. The microphone 8 converts sound from the user or another source to an audio signal and transmits the audio signal to the controller 10. The smartphone 1 may include a speaker instead of the receiver 7.

The storage 9 functions as a memory storing programs and data. The storage 9 may also be used as a memory for storing results of processing by the controller 10 temporarily. The storage 9 may include any appropriate storage device, such as a semiconductor storage device or a magnetic storage device. The storage 9 may also include a plurality of types of storage devices. The storage 9 may include a combination of a portable storage medium, such as a memory card, and an apparatus for reading the storage medium.

The programs stored on the storage 9 include applications that run in the foreground or the background and a control program that supports operations of the applications. The applications may, for example, display a predetermined screen on the display 2A and cause the controller 10 to execute processing in accordance with a gesture detected by the touchscreen 2B. The control program may, for example, be an operating system (OS). The applications and the control program may be installed on the storage 9 through wireless communication by the communication interface 6 or from a storage medium.

The storage 9 for example stores a control program 9A, a mail application 9B, a browser application 9C, and a measurement application 9Z. The mail application 9B provides e-mail functions for actions such as creating, sending, receiving, and displaying e-mail. The browser application 9C provides a Web browsing function to display Web pages. The measurement application 9Z provides a function for the user of the smartphone 1 to measure the contour of a cross-section of an object.

The control program 9A provides functions related to various types of control which enable the smartphone 1 to operate. The control program 9A may, for example, place a phone call by controlling components such as the communication interface 6, receiver 7, and microphone 8. The functions provided by the control program 9A may be used in combination with functions provided by other programs, such as the mail application 9B.

The controller 10 may, for example, be a central processing unit (CPU). The controller 10 may be a system-on-a-chip (SoC) or other type of integrated circuit in which other components, such as the communication interface 6, are integrated. The controller 10 may be configured by combining a plurality of integrated circuits. The controller 10 functions as a control unit for implementing a variety of functions by comprehensively controlling operations of the smartphone 1.

Specifically, the controller 10 refers as necessary to data stored in the storage 9. The controller 10 executes commands included in the programs stored in the storage 9 to control components such as the display 2A, the communication interface 6, and the motion sensor 15, thereby implementing various functions. The controller 10 implements various functions by executing commands included in the measurement application 9Z stored in the storage 9. The controller 10 can change the control in response to detection results from various detectors, such as the touchscreen 2B, buttons 3, and motion sensor 15. In the present embodiment, the entire controller 10 functions as a control unit. The controller 10 calculates a contour of the cross-section of an object based on orientation information acquired by the first sensor and movement information acquired by the second sensor.

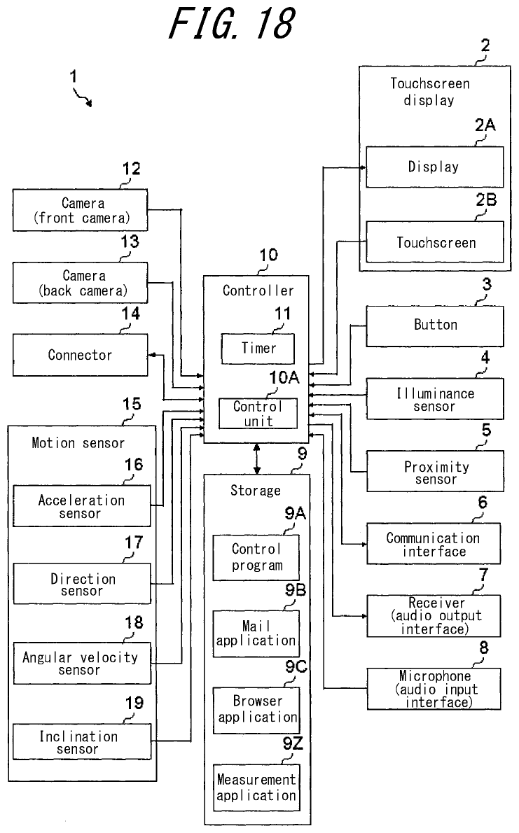

The timer 11 outputs a clock signal with a preset frequency. The timer 11 receives an instruction for a timer operation from the controller 10 and outputs the clock signal to the controller 10. The first sensor and the second sensor acquire orientation information and movement information multiple times in accordance with clock signals input through the controller 10. The timer 11 may be provided externally to the controller 10 or may be included in the controller 10, as illustrated below in FIG. 18.

The camera 12 is a front camera that images an object facing the front face 1A. The camera 13 is a back camera that images an object facing the back face 1B.

The connector 14 is a terminal to which another apparatus connects. The connector 14 of the present embodiment also functions as a communication interface for communication between the smartphone 1 and another apparatus over a connection object connected to the terminal. The connector 14 may be a general-purpose terminal such as a universal serial bus (USB), high-definition multimedia interface (HDMI® (HDMI is a registered trademark in Japan, other countries, or both)), mobile high-definition link (MHL), Light Peak, Thunderbolt, local area network connector, or an earphone microphone connector. The connector 14 may be designed as a dedicated terminal, such as a dock connector. Examples of the apparatuses that connect to the connector 14 include a charger, an external storage, a speaker, a communication apparatus, and an information processing apparatus.

The motion sensor 15 detects a motion factor. This motion factor is mainly processed as a control factor of the smartphone 1, i.e. the electronic device. The control factor is a factor indicating the status of the electronic device and is processed by the controller 10. The motion sensor 15 functions as a measurement unit for measuring the contour of the user's abdomen. The measurement unit may include the above-described cameras 12 and/or 13. The motion sensor 15 of the present embodiment includes an acceleration sensor 16, a direction sensor 17, an angular velocity sensor 18, and an inclination sensor 19. The combined output of the acceleration sensor 16, direction sensor 17, angular velocity sensor 18, and inclination sensor 19 can be used. By processing the combined output of the motion sensor 15, the controller 10 can execute processing that amply reflects the movement of the smartphone 1, i.e. the electronic device.

In the present embodiment, the first sensor obtains the orientation information of the smartphone 1, i.e. the electronic device. The orientation information of the smartphone 1 is outputted from the first sensor. The orientation information of the smartphone 1 is related to the direction in which the smartphone 1 is facing. The orientation information of the smartphone 1 for example includes the direction of the earth's magnetism, the inclination relative to the earth's magnetism, the direction of the rotation angle, the change in the rotation angle, the direction of gravity, and the inclination relative to the direction of gravity.

The orientation of the smartphone 1 refers to the direction of a normal to the surface of the housing 20 that is opposite the object when the contour of the cross-section of the object is being measured. The surface of the housing 20 that is opposite the object may be any surface whose orientation can be detected by the first sensor. This surface may be any of the front face 1A, the back face 1B, and the side faces 1C1 to 1C4.

In the present embodiment, the direction sensor 17 is used as the first sensor. The direction sensor 17 is a sensor that detects the orientation of the earth's magnetism. In the present embodiment, the component when the orientation of the smartphone 1 is projected onto a plane parallel to the ground is the orientation information acquired by the direction sensor 17. The orientation information acquired by the direction sensor 17 is the direction of the smartphone 1. The direction of the smartphone 1 can be acquired as 0° to 360° orientation information. For example, the orientation information that is acquired is 0° when the smartphone 1 is facing north, 90° when facing east, 180° when facing south, and 270° when facing west. In the present embodiment, the direction sensor 17 can more accurately acquire the orientation information as a result of a cross-section of the measured object being parallel to the ground. When the object is the abdomen, the contour of the abdomen can be measured while the user is standing.

The direction sensor 17 outputs the detected orientation of the earth's magnetism. For example, when the orientation of the earth's magnetism is output as a motion factor, the controller 10 can execute processing using this motion factor as a control factor that reflects the direction in which the smartphone 1 faces. For example, when the change in the orientation of the earth's magnetism is output as a motion factor, the controller 10 can execute processing using this motion factor as a control factor that reflects the change in the orientation of the smartphone 1.

The angular velocity sensor 18 may be used as the first sensor. The angular velocity sensor 18 detects the angular velocity of the smartphone 1. The angular velocity sensor 18 can acquire the angular velocity of the smartphone 1 as orientation information. The controller 10 calculates the orientation of the smartphone 1 by time integrating the acquired angular velocity once. The calculated orientation of the smartphone 1 is an angle relative to an initial value at the start of measurement.

The angular velocity sensor 18 outputs the detected angular velocity. For example, when the orientation of the angular velocity is output as a motion factor, the controller 10 can execute processing using this motion factor as a control factor that reflects the rotation direction of the smartphone 1. For example, when the magnitude of the angular velocity is output, the controller 10 can execute processing using this magnitude as a control factor that reflects the rotation amount of the smartphone 1.

The inclination sensor 19 may be used as the first sensor. The inclination sensor 19 detects the gravitational acceleration acting on the smartphone 1. The inclination sensor 19 can acquire the gravitational acceleration of the smartphone 1 as orientation information. For example, with the inclination sensor 19, the smartphone 1 can acquire −9.8 m/s² to 9.8 m/s² as the orientation information. The acquired orientation information is 9.8 m/s² when, for example, the y-axis direction of the smartphone 1 illustrated in FIG. 1 is the same as the direction of gravity and is −9.8 m/s² in the opposite case. When the y-axis direction is perpendicular to the direction of gravity, the acquired orientation information is 0 m/s². In the present embodiment, the inclination sensor 19 can more accurately acquire the orientation information as a result of a cross-section of the measured part being perpendicular to the ground. When the object is the abdomen, the contour of the abdomen can be measured while the user is lying down.

The inclination sensor 19 outputs the detected inclination. For example, when the inclination relative to the direction of gravity is output as a motion factor, the controller 10 can execute processing using this motion factor as a control factor that reflects the inclination of the smartphone 1.

In some cases, the controller 10 calculates the orientation based on the orientation information of the smartphone 1. For example, the above-described angular velocity sensor 18 acquires the angular velocity as orientation information. Based on the acquired angular velocity, the controller 10 calculates the orientation of the smartphone 1. As another example, the above-described inclination sensor 19 acquires the gravitational acceleration as orientation information. Based on the acquired gravitational acceleration, the controller 10 calculates the orientation of the smartphone 1 relative to the direction of gravity.

A combination of motion sensors 15 described above can be used as the first sensor. By processing a combination of orientation information from a plurality of motion sensors, the controller 10 can more accurately calculate the orientation of the smartphone 1, i.e. the electronic device.

In the present embodiment, the device for obtaining movement information of the electronic device is the second sensor. The second sensor obtains movement information of the smartphone 1, i.e. the electronic device. The movement information of the smartphone 1 is outputted from the second sensor. The movement information of the smartphone 1 is related to the movement amount of the smartphone 1. The movement information of the smartphone 1 for example includes acceleration, speed, and movement amount.

In the present embodiment, the movement amount of the smartphone 1 is the movement amount of a reference position of the housing 20 in the smartphone 1. The reference position of the housing 20 may be any position detectable by the second sensor, such as the surface of the side face 1C1.

In the present embodiment, the acceleration sensor 16 is used in the second sensor. The acceleration sensor 16 detects the acceleration acting on the smartphone 1. The acceleration sensor 16 can acquire the acceleration of the smartphone 1 as movement information. The controller 10 calculates the movement amount of the smartphone 1 by time integrating the acquired acceleration twice.

The acceleration sensor 16 outputs the detected acceleration. For example, when the direction of the acceleration is output, the controller 10 can execute processing using this direction as a control factor that reflects the direction in which the smartphone 1 is moving. For example, when the magnitude of the acceleration is output, the controller 10 can execute processing using this magnitude as a control factor that reflects the speed at which the smartphone 1 is moving and the movement amount.

The controller 10 calculates the contour of a cross-section of the object. The contour of the cross-section of the object is calculated based on the orientation information and movement information acquired by the first sensor and the second sensor. In some cases, the controller 10 calculates the orientation and the movement amount during the calculation process.

A sensor that can detect motion factors in three axial directions is used in the above-described motion sensor 15. The three axial directions detected by the motion sensor 15 of the present embodiment are substantially orthogonal to each other. The x-direction, y-direction, and z-direction illustrated in FIGS. 1 to 3 correspond to the three axial directions of the motion sensor 15. The three axial directions need not be orthogonal to each other. In a motion sensor 15 in which the three directions are not orthogonal to each other, motion factors in three orthogonal directions can be calculated. The direction serving as a reference may differ for each motion sensor 15. In the present embodiment, each motion sensor 15 is not necessarily a three-axis sensor. The controller 10 can calculate the contour of a cross-section with the orientation information in one axial direction and the movement information in one axial direction.

The first sensor and the second sensor may use any of the above-described motion sensors 15 or another motion sensor.

A portion or all of the programs stored in the storage 9 in FIG. 4 may be downloaded by the communication interface 6 from another apparatus by wireless communication. A portion or all of the programs stored in the storage 9 in FIG. 4 may also be stored in a non-transitory storage medium that is readable by a reading apparatus included in the storage 9. A portion or all of the programs stored in the storage 9 in FIG. 4 may also be stored in a non-transitory storage medium that is readable by a reading apparatus connected to the connector 14. Examples of the storage medium include flash memory, a hard disk drive (HDD®), a compact disk (CD), a digital versatile disc (DVD®), and a Blu-ray® disc (HDD, DVD, and Blu-ray are registered trademarks in Japan, other countries, or both).

The configuration of the smartphone 1 illustrated in FIGS. 1 to 4 is only an example and may be changed as necessary without departing from the scope of the present disclosure. For example, the number and type of buttons 3 are not limited to the example in FIG. 1. Instead of including the buttons 3A to 3C, for example, the smartphone 1 may include buttons arranged as a numeric keypad, a QWERTY keyboard, or another arrangement as buttons for operating the screen. In order to operate the screen, the smartphone 1 may include just one button or may lack buttons altogether. In the example in FIG. 4, the smartphone 1 includes two cameras, but the smartphone 1 may include just one camera or may lack cameras altogether. The illuminance sensor 4 and the proximity sensor 5 may be configured by one sensor. In the example illustrated in FIG. 4, four types of sensors are provided to acquire the orientation information and the movement information of the smartphone 1, i.e. the electronic device. The smartphone 1 need not include all of these sensors, however, and may include other types of sensors.

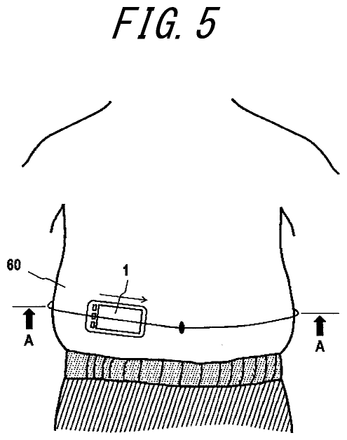

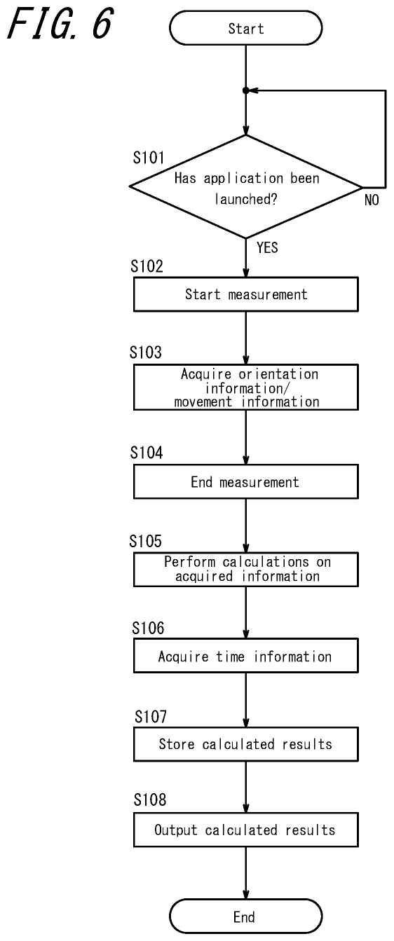

Next, with reference to FIGS. 5 and 6, measurement of the contour of an abdominal cross-section by the smartphone 1 according to an embodiment is described.

FIG. 5 is a schematic diagram illustrating measurement of the contour of an abdominal cross-section according to an embodiment.

FIG. 6 is a flowchart for measurement of the contour of an abdominal cross-section according to an embodiment.

In step S101, the user launches the measurement application 9Z for measuring the contour of a cross-section. Next, measurement begins in step S102. At the start of measurement, the smartphone 1 is placed against the surface of the abdomen 60 at any position where the contour of an abdominal cross-section is to be measured. In the present embodiment, the contour of a cross-section at the height of the user's navel (the position indicated by A-A in

FIG. 5) is measured. As long as measurement of the contour of the cross-section is not impeded, the smartphone 1 may be contacted to the surface of the abdomen 60 directly or with clothing therebetween. The measurement start position may be anywhere along the abdominal A-A position. To start measurement, the user performs a preset start action on the smartphone 1. The preset start action may be an action such as pushing one of the buttons 3 of the smartphone 1 or tapping a particular position on the touchscreen 2B. The opposing face placed against the surface of the abdomen may be any of the front face 1A, back face 1B, and side faces 1C1 to 1C4 of the smartphone 1. For operability, however, the back face 1B is the opposing face in the present embodiment.

In step S103, the user moves the smartphone 1 along the surface at the A-A position of the abdomen 60 once around the abdomen 60. If the user moves the smartphone 1 at a constant speed while keeping the smartphone 1 against the surface of the abdomen 60, the interval between acquisition of various information becomes constant, which increases the accuracy of contour measurement.

In step S103, under conditions programmed in advance, the direction sensor 17 acquires orientation information and the acceleration sensor 16 acquires movement information. The orientation information and movement information are acquired multiple times. The orientation information and the movement information are acquired in accordance with the clock signal output from the timer 11. The acquisition cycle for each type of information may be selected in accordance with the size and/or complexity of the cross-section of the measured object. The acquisition cycle of information may, for example, be selected from among a sampling frequency of 5 Hertz (Hz) to 60 Hz. The acquired orientation information and movement information are temporarily stored inside the smartphone 1. This measurement is continuously made from the start of step S102 until the end of step S104.

After moving the smartphone 1 once around the abdomen 60 while keeping the smartphone 1 against the abdomen 60, the user performs an end action, set in advance, on the smartphone 1 to end measurement (step S104). The end action set in advance may be an action such as pushing one of the buttons 3 of the smartphone 1 or tapping a particular position on the touchscreen 2B. Alternatively, the smartphone 1 may automatically end measurement by recognizing one circumference when the orientation information acquired by the direction sensor 17 of the smartphone 1 matches the orientation information at the start of measurement or changes by 360° from the orientation information at the start of measurement. In the case of automatic recognition, the user need not perform the end action, thereby simplifying measurement.

In step S105, calculations are performed on the orientation information and the movement information acquired in step S103. The controller 10 performs these calculations. The controller 10 calculates the contour and girth of the cross-section of the user's abdomen. Details on the calculations in step S105 are provided below.

In step S106, the smartphone 1 acquires information related to the time at which measurement was performed (“time information”). The smartphone 1 acquires the time information using a clock function, such as a real-time clock (RTC). The time information is not necessarily acquired after the calculations in step S105. The time information may, for example, be acquired at any timing related to measurement, such as after the application is launched in step S101, or after the end of measurement in step S104.

In step S107, the smartphone 1 associates the result of calculations in step S105 with the time information acquired in step S106 and stores the associated result and time information in the storage 9.

In step S108, the smartphone 1 may output the result of calculations performed in step S105 (the contour and girth of the cross-section of the user's abdomen). Examples of the method of outputting the calculated results include displaying the results on the display 2A and transmitting the results to a server. The smartphone 1 may output the calculated results so as to be displayed in overlap with the contour and girth of the cross-section of the abdomen measured at a different time. Details of how the smartphone 1 displays the contour and girth of the cross-section of the abdomen are provided below. Once output of the results of calculating the contour and girth of the cross-section of the abdomen is complete, the smartphone 1 ends the processing flow.

In the present embodiment, the back face 1B of the smartphone 1 is placed against the abdomen and moved in the y-axis direction. In this case, it suffices for the direction sensor 17 to be a uniaxial sensor capable of measuring the orientation in the y-axis direction of the smartphone 1. It suffices for the acceleration sensor 16 to be a uniaxial sensor capable of measuring the movement amount in the y-axis direction.

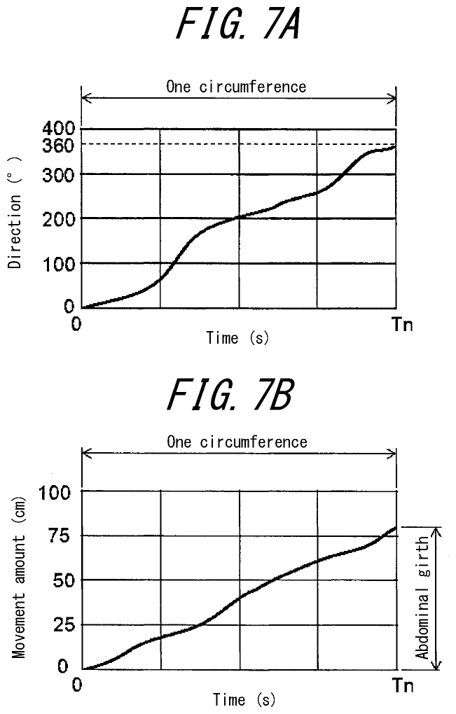

Next, the method of calculating the contour of the cross-section is described with reference to FIGS. 7A to 9, taking the smartphone 1 as an example.

FIGS. 7A and 7B illustrate an example of orientation and movement amount according to an embodiment.

The horizontal axis in FIGS. 7A and 7B indicates the time from the start to the end of measurement. Time is counted by the clock signal output by the timer 11. When the circumference of the abdomen is measured in Tn seconds (s), the start of measurement is at 0 s and the end of measurement at Tn s. Over predetermined acquisition cycles, the smartphone 1 acquires the orientation information and movement information from 0 s to Tn s.

In FIG. 7A, the horizontal axis represents time, and the vertical axis represents the direction of the smartphone 1. The direction of the smartphone 1 on the horizontal axis is orientation information acquired by the direction sensor 17. The direction sensor 17 is adopted as the first sensor in the present embodiment. Hence, the orientation information is the direction of the smartphone 1. The direction of the smartphone 1 is represented as an angle from 0° to 360°. The direction of the smartphone 1 is determined to have completed one circumference upon changing 360° from the initial orientation of measurement. In the present embodiment, the initial orientation of measurement is set to 0° for ease of understanding, making the orientation 360° after one circumference.

In FIG. 7B, the horizontal axis represents time, and the vertical axis represents the movement amount of the smartphone 1. The movement amount of the smartphone 1 on the vertical axis is calculated based on the movement information acquired by the acceleration sensor 16. The movement information of the smartphone 1 in the present embodiment is acceleration data acquired by the acceleration sensor 16. The movement amount is calculated by the controller 10 by time integrating the acceleration data twice. When the acceleration data includes a large amount of noise, digital filtering may be performed. The digital filter may, for example, be a low pass filter or a band pass filter. The movement amount of the smartphone 1 at the end of measurement corresponds to the circumference of the measured object, i.e. the abdominal girth in the present embodiment. The abdominal girth may be calculated taking into account the arrangement of the acceleration sensor 16 within the smartphone 1. In other words, the abdominal girth may be calculated accurately in the present embodiment by correction of the movement amount taking into consideration the interval between the acceleration sensor 16 and the back face 1B, which is the opposing surface placed against the surface of the abdomen 60.

In the present embodiment, the case of measuring direction and the movement amount during the same time Tn has been illustrated, but the direction and the movement amount may be measured in different times Ta and Tb. In that case, the horizontal axis of FIG. 7A may use a normalized time 0-1 normalized by Ta, the horizontal axis of FIG. 7B may use a normalized time 0-1 normalized by Tb, and the numerical values on each horizontal axis may be aligned.

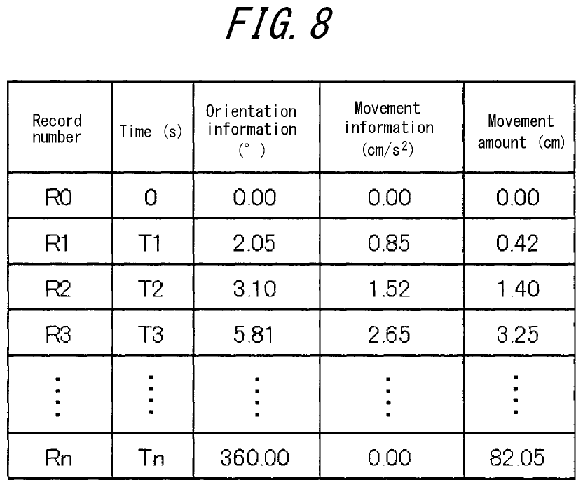

FIG. 8 is an example record formed by acquired information.

The record number at the start of measurement is R0, and the record number at the end of measurement is Rn. In each record, orientation information and movement information corresponding to time are stored as a pair. Furthermore, the movement amount calculated based on the movement information is stored in each record. In the present embodiment, which uses the direction sensor 17, the orientation information is the direction faced by the smartphone 1. The direction and movement amount, which are information calculated based on the pair of orientation information and movement information, are acquired at the same time in FIGS. 7A and 7B. The direction and movement amount, which are information calculated based on the pair of orientation information and movement information, may be acquired at the same standardized time. The time intervals between the records need not be equal intervals. A pair of records may be information acquired at the same time, or the acquisition times may differ. When the acquisition times differ, the controller 10 may take the time difference into account.

FIG. 9 illustrates a calculated contour of a cross-section.

The contour of the cross-section of the object can be calculated by plotting the acquired records R0 to Rn in order in accordance with orientation and movement amount. The labels R0 to Rn in FIG. 9 indicate the corresponding record numbers. The points on the solid line indicate the positions of the records. The line actually includes many more points, but some of the points are omitted to clarify the drawing.

The contour of a cross-section is calculated as follows. First, R0 is set at any point. Next, the position of R1 is calculated from the amount of change in the movement amount between record R0 and record R1 and the orientation information of record R1. Next, the position of R2 is calculated from the amount of change in the movement amount between record R1 and record R2 and the orientation information of record R2. This calculation is made up to Rn. By connecting the positions in order from the position of R0 to the position of Rn, the contour of the cross-section of the object is calculated and then displayed.

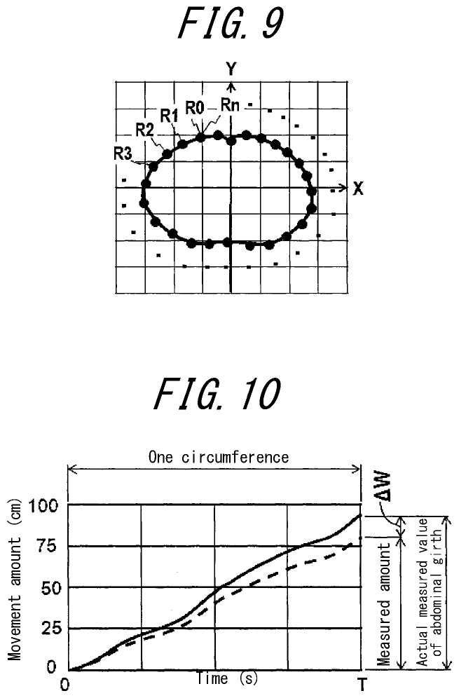

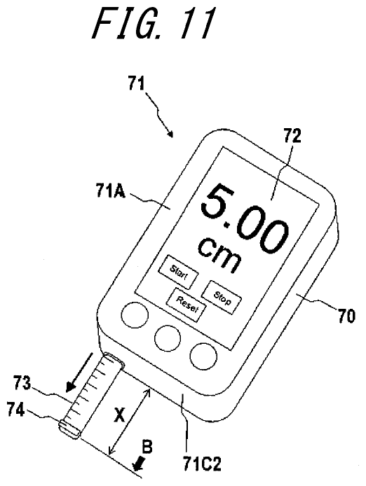

FIG. 10 illustrates correction using an actual measured value according to an embodiment.

In the above embodiment, the movement information acquired by the acceleration sensor 16 is used to calculate the contour of the cross-section. The actual measured circumference of the object as measured in advance by other means, however, may be used. In FIG. 10, the horizontal axis represents time, and the vertical axis represents the movement amount. The dotted line in FIG. 10 is the movement amount calculated based on the movement information acquired by the acceleration sensor 16. The movement amount at the end of measurement corresponds to the circumference of the measured object. In the present embodiment, the movement amount corresponds to the abdominal girth. The movement amount at the end of measurement is corrected so as to equal the abdominal girth actually measured in advance by a tape measure or other instrument. In greater detail, the movement amount at the end of measurement is offset by the correction amount ΔW in FIG. 10, and the inclination of the graph is corrected to match the movement amount offset by ΔW. The corrected data is indicated by a solid line. The controller 10 calculates the contour of the cross-section of the object using the records that include the corrected, solid-line data.

Next, correction of the orientation and position of the calculated contour of a cross-section is described. Upon setting the orientation of the smartphone 1 at the start of measurement to 0°, the axis of symmetry of the calculated contour of a cross-section might be inclined. For example, in the case of the contour of an abdominal cross-section, the user may wish to correct the inclination and display the contour with the abdomen or the back directly facing the y-axis direction in FIG. 9. On the coordinate axes of FIG. 9, the inclination may be corrected by rotating the contour of the cross-section so that the width in the x-axis direction of the contour or the width in the y-axis direction of the contour is minimized or maximized.

If the position coordinates of the smartphone 1 at the start of measurement are at the xy origin in FIG. 9, the calculated contour of a cross-section is displayed as being shifted from the center. The user may wish for the xy origin in FIG. 9 and the center of the contour of the abdominal cross-section to coincide when the contour of the abdominal cross-section is displayed. The center of the contour of the abdominal cross-section may be considered the intersection of the widest center line of the contour of the cross-section in the x-axis direction and the widest center line of the contour of the cross-section in the y-axis direction. Furthermore, the center of the contour of the abdominal cross-section may be moved to the xy origin of FIG. 9.

As described above, in a device according to the present embodiment, the contour of the cross-section of the object can be measured by a sensor built into the smartphone 1. The smartphone 1 is smaller than a measurement apparatus such as a CT apparatus. The smartphone 1 can also rapidly measure the contour of a cross-section. Users of the smartphone 1 can measure data themselves, thereby simplifying measurement. The smartphone 1 can be carried easily, which is not true of CT apparatuses and the like. Since users of the smartphone 1 can measure data themselves, they can easily recognize day-to-day changes. The smartphone 1 also entails little risk of radiation exposure during measurement.

FIG. 11 schematically illustrates an electronic tape measure according to an embodiment.

An electronic tape measure has a function to measure the length of extracted tape and acquire data. Hence, an electronic tape measure can acquire movement information like the acceleration sensor 16. The electronic tape measure may also be built into the smartphone 1.

An electronic tape measure 71 includes a housing 70. A touchscreen display 72 is provided on a front face 71A of the housing 70. A tape measure 73 is provided on the side face 71C2 of the housing 70. Measurement markings are inscribed on the tape measure 73. The tape measure 73 is normally wound up inside the housing 70. A stopper 74 is provided at the end of the tape measure 73. Before measurement, the stopper 74 is placed outside of the housing 70, and the B face of the stopper 74 is in contact with the side face 71C2. To measure a dimension of the object, the stopper 74 is pulled in the direction of the arrow in FIG. 11 to extract the tape measure 73 from the housing 70. At this time, the extracted amount X of the tape measure 73 with reference to the side face 71C2 is digitally displayed on the touchscreen display 72. The embodiment in FIG. 11 illustrates the case of X=5.00 cm.

In the case of the electronic tape measure 71 being used as the second sensor of the smartphone 1 in the present embodiment, the measurement procedure and the calculation of the contour of the cross-section conform to the description of FIGS. 5 through 9. The measurement procedure when the electronic tape measure 71 is used is described below. At the start of measurement in step S102, the housing 70 is placed against the surface of the abdomen. In step S103, the user moves the housing 70 along the surface at the A-A position of the abdomen 60 around the abdomen 60 once while holding the stopper 74 at the measurement start position. Measurement ends when the side face 71C2 and the B face of the stopper 74 coincide (step S104).

When the acceleration sensor 16 is used as the second sensor, the acceleration is acquired as the movement information. By contrast, when the electronic tape measure 71 is used as the second sensor, the length is acquired directly as the movement information. Use of the electronic tape measure 71 as the second sensor thus allows more accurate measurement of the abdominal girth.

Next, the method by which the user uses the smartphone 1 and the data stored in the storage 9 of the smartphone 1 are described.

The storage 9 of the smartphone 1 stores a contour of a cross-section of the user's abdomen, measured by the above-described method, in association with the time at which the contour was measured. By the method described with reference to FIG. 6, for example, the smartphone 1 can store a contour in association with the time at which the contour was measured in the storage 9.

The storage 9 of the smartphone 1 stores information related to food or drink consumed by the user in association with the time at which the food or drink was consumed. The information related to food or drink may, for example, include at least one of the type, the amount, and the number of calories of the food or drink. The information related to food or drink may, for example, include at least one of the name of the food or drink and the raw materials and ingredients (such as nutrients) included in the food or drink. Food or drink in this context may include any of general food items, health food, and medicine.

The smartphone 1 can acquire information related to food or drink by various methods. For example, the smartphone 1 can acquire information related to food or drink by receiving input from the user to the touchscreen 2B and/or the buttons 3. In this case, the user uses the touchscreen 2B and/or the buttons 3 to input information related to food or drink directly to the smartphone 1. The user inputs information related to food or drink when consuming the food or drink, for example. The controller 10 of the smartphone 1 stores the time at which the information related to food or drink was inputted in the storage 9 in association with the information related to food or drink.

The smartphone 1 may acquire information related to food or drink based on information included in the food or drink package, for example. The information included in the food or drink package includes a barcode, or Japanese article number (JAN), for example. When a barcode is listed on the food or drink package, the user photographs the barcode using the camera 13 of the smartphone 1. The controller 10 of the smartphone 1 scans the photographed barcode and stores information related to the product associated with the barcode in the storage 9 as the information related to food or drink. At this time, the controller 10 may acquire the information related to the product associated with the barcode by communicating with an external information processing apparatus, for example. The user scans the barcode using the smartphone 1 when consuming the food or drink, for example. The controller 10 of the smartphone 1 stores the time at which the barcode was scanned in the storage 9 in association with the information related to food or drink. When codes other than the barcode (for example, a one-dimensional code or two-dimensional code) are included on the food or drink package, the smartphone 1 may acquire the information related to food or drink by scanning the other codes.

The information related to food or drink includes a radio frequency identifier (RFID), for example. Suppose that an RFID tag with information related to food or drink is provided in the food or drink package, and the smartphone 1 is an electronic device supporting RFID. In this case, the user causes the smartphone 1 to acquire the information related to food or drink from the RFID tag provided in the food or drink package. The controller 10 of the smartphone 1 stores the acquired information related to food or drink in the storage 9. The controller 10 of the smartphone 1 stores the time at which the information related to food or drink was acquired by RFID communication in the storage 9 in association with the information related to food or drink.

The information related to food or drink includes information related to nutritional information listed on a package, for example. When information related to nutritional information is listed on a food or drink package, for example, the user photographs the nutritional information column on the package using the camera 13 of the smartphone 1. The controller 10 of the smartphone 1 reads the photographed nutritional information column and stores the information listed as nutritional information (in this case, the number of calories, the nutrients included in the food product and the amount thereof, and the like) in the storage 9 as the information related to food or drink. At this time, the controller 10 may communicate with an external information processing apparatus, for example, and transmit the captured image to the external information processing apparatus. In this case, the external information processing apparatus reads the photographed nutritional information column and transmits the information listed as nutritional information to the smartphone 1. The smartphone 1 stores the acquired information in the storage 9 as information related to food or drink. The controller 10 of the smartphone 1 stores the time at which the nutritional information column was photographed in the storage 9 in association with the information related to food or drink.

The information related to food or drink may be estimated based on an image of the food or drink. For example, the user uses the camera 13 of the smartphone 1 to photograph an image of the food or drink before consumption. The controller 10 estimates the information related to food or drink by analyzing the photographed image. The controller 10 can perform image analysis to estimate the amount of the food or drink based on the volume of the food or drink, for example. The controller 10 can, for example, perform image analysis to estimate the nutrients included in the food or drink based on the color of the food or drink. The color of the ingredients in the food or drink does not necessarily correspond to the nutrients included in the ingredients. The nutrients can be estimated, however, from the color of the ingredients. The controller 10 may estimate the calories in the food or drink based on the photographed image. The controller 10 stores the estimated information in the storage 9 as the information related to food or drink. The controller 10 stores the time at which the image of the food or drink was captured in the storage 9 in association with the information related to food or drink.

Estimation of the information related to food or drink based on the photographed image of the food or drink is not necessarily made by the controller 10 of the smartphone 1. For example, the controller 10 of the smartphone 1 may transmit the photographed image of the food or drink to an external information processing apparatus. The external information processing apparatus estimates the information related to food or drink based on the image of the food or drink. The external information processing apparatus then transmits the estimated information related to food or drink to the smartphone 1. The smartphone 1 stores the information related to food or drink acquired from the external information processing apparatus in the storage 9.

In addition to the image of the food or drink before consumption, the user may also capture an image of the food or drink after consumption using the camera 13 of the smartphone 1. In this case, the controller 10 or the external information processing apparatus can estimate the content of the user's leftover food or drink based on the image of the food or drink after consumption. The controller 10 or the external information processing apparatus can therefore more easily estimate the information related to food or drink for the food or drink actually consumed by the user.

The storage 9 of the smartphone 1 stores information related to the user's physical activity in association with the time at which the physical activity was performed. In the present disclosure, the information related to physical activity refers to activity performed as part of the user's life. The information related to physical activity may, for example, include information related to exercise and information related to sleep. The information related to exercise may, for example, include at least one of the amount of exercise and calories burned. In the present disclosure, the amount of exercise may include the content and duration of exercise. The information related to sleep may include the hours of sleep.

The smartphone 1 can acquire information related to exercise by various methods. For example, the smartphone 1 can acquire information related to exercise by receiving input from the user to the touchscreen 2B and/or the buttons 3. In this case, the user uses the touchscreen 2B and/or the buttons 3 to input information related to exercise directly to the smartphone 1. The user may, for example, input information related to exercise before or after performing exercise. Based on user input, the controller 10 of the smartphone 1 stores the time at which the user performed exercise in the storage 9 in association with the information related to exercise.

The smartphone 1 may estimate the information related to exercise based on information acquired by a sensor provided in the electronic device. For example, when the user is wearing the smartphone 1 while exercising, the controller 10 of the smartphone 1 estimates the information related to exercise, such as the intensity and duration of exercise, based on the magnitude of the user's body movements detected by the motion sensor 15. The controller 10 judges that the user is exercising when, for example, the magnitude of body movements exceeds a predetermined body movement threshold. The controller 10 estimates the duration of exercise by the user as the length of time that the magnitude of body movements continuously exceeds the predetermined body movement threshold. The controller 10 can estimate the starting time of exercise as the time when the magnitude of body movements exceeds the threshold and the ending time as the time when the magnitude of body movements falls below the threshold. The controller 10 may set a plurality of predetermined body movement thresholds and estimate the starting time and ending time of exercise corresponding to exercise at a plurality of intensity levels. The controller 10 may count the number of steps based on the user's body movements and calculate the calories burned from the number of steps.

When the smartphone 1 includes a sensor capable of detecting biological information, such as the user's pulse or body temperature, the controller 10 may estimate the information related to exercise based on the biological information. A person's pulse increases during exercise, and the body temperature rises. Predetermined exercise judgment thresholds may be set in advance for the pulse and body temperature to judge whether the user is exercising. The controller 10 can estimate the starting time of exercise as the time at which the pulse and body temperature exceed the predetermined exercise judgment thresholds. The controller 10 can also estimate the user's exercise intensity based on changes in the pulse and body temperature.

After estimating the information related to exercise, the controller 10 stores the time at which the user performed exercise in the storage 9 in association with the estimated information related to exercise. The time at which the user performed exercise may be either or both of the exercise starting time and the exercise ending time.

The information related to exercise is not necessarily estimated by the controller 10 of the smartphone 1. For example, an information processing apparatus external to the smartphone 1 may estimate the information related to exercise and transmit the estimated information related to exercise to the smartphone 1. The controller 10 of the smartphone 1 stores the information related to exercise acquired from the external information processing apparatus in the storage 9.

The information used to estimate the information related to exercise is not necessarily acquired by the smartphone 1. For example, information from the user may be acquired by a dedicated electronic device that differs from the smartphone 1 and includes a motion sensor capable of detecting the user's body movements or a biological sensor capable of acquiring biological information of the user. In this case, the information acquired by the dedicated electronic device may be transmitted to the smartphone 1 or the external information processing apparatus, and information related to exercise may be estimated on the smartphone 1 or the external information processing apparatus.

The smartphone 1 can acquire information related to sleep by various methods. For example, the smartphone 1 can acquire the information related to sleep by receiving input from the user to the touchscreen 2B and/or the buttons 3. In this case, the user uses the touchscreen 2B and/or the buttons 3 to input the information related to sleep directly to the smartphone 1. The user can input the information related to sleep by operating the smartphone 1 before going to bed or after getting up, for example. Based on user input, the controller 10 of the smartphone 1 stores the time related to the user's sleep (such as the time the user falls asleep) in the storage 9 in association with the information related to sleep.

The smartphone 1 may infer the information related to sleep based on information acquired by a sensor provided in the electronic device. For example, when the user is wearing the smartphone 1 while sleeping, the controller 10 of the smartphone 1 infers the information related to sleep based on the user's body movements detected by the motion sensor 15. The inference of information related to sleep by the controller 10 is now described in detail. It is known that people repeatedly experience two sleeping states, REM sleep and non-REM sleep, over a nearly constant cycle while sleeping. People are more likely to turn over during REM sleep and less likely to turn over during non-REM sleep. The controller 10 uses these tendencies to infer the user's sleep state based on body movements caused by the user turning over. In other words, the controller 10 infers that the time period when body movements are detected in a predetermined cycle is non-REM sleep and infers that the time period when body movements are not detected in a predetermined cycle is REM sleep. The two sleep states are repeated over a nearly constant cycle. Therefore, after determining the cycle of the two sleep states, the controller 10 can calculate backwards to infer the time at which the user actually fell asleep based on the time periods of the two sleep states.

When the smartphone 1 includes a sensor capable of detecting biological information, such as the user's pulse or body temperature, the controller 10 may estimate the information related to exercise based on the biological information. When people sleep, their pulse lowers, and their body temperature falls. The controller 10 can set predetermined sleep judgment thresholds in advance for the pulse and body temperature and estimate the actual time at which the user falls asleep as the time when the pulse and body temperature fall below the predetermined sleep judgment thresholds.

Like the information related to exercise, the information related to sleep may also be estimated by an external information processing apparatus. Furthermore, like the information related to exercise, the information related to sleep may also be estimated based on information acquired by a dedicated electronic device.

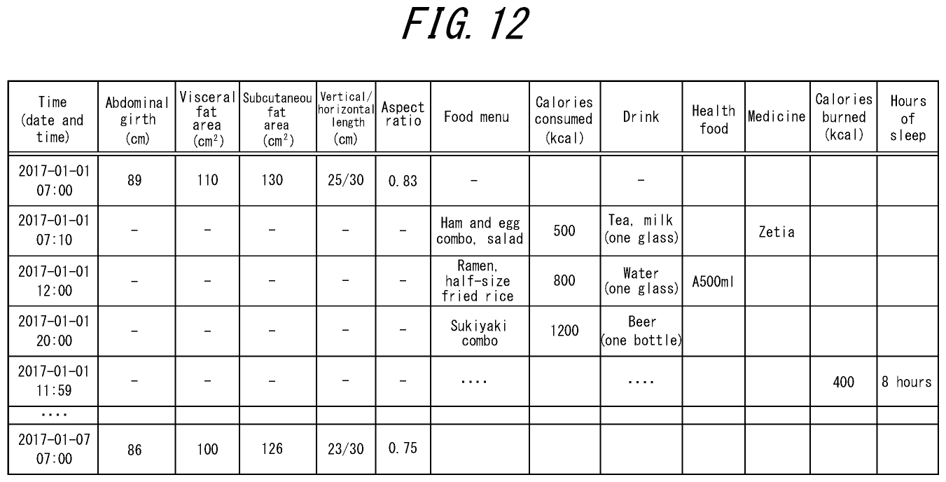

FIG. 12 illustrates example data stored in the storage 9 of the smartphone 1. As illustrated in FIG. 12, various information is stored in the storage 9 in association with time (date and time). For example, the contour of an abdominal cross-section measured by the smartphone 1 and information related thereto are stored in the storage 9 in association with time. The information related to the contour of the abdominal cross-section may, for example, include the abdominal girth, the visceral fat area, the subcutaneous fat area, the vertical/horizontal length, and the aspect ratio, as illustrated in FIG. 12.

The abdominal girth is calculated by the method described with reference to FIG. 6. The abdominal girth may be inputted by the user.

The visceral fat area and the subcutaneous fat area are, for example, estimated based on the calculated contour of the abdominal cross-section. The method by which the smartphone 1 estimates the visceral fat area and the subcutaneous fat area is now described. For example, the storage 9 stores estimation formulas, derived in advance, for the visceral fat area and the subcutaneous fat area. The controller 10 extracts characteristic coefficients of the contour of the cross-section of the object calculated as described above. The controller 10 reads the estimation formulas, stored in the storage 9, for the visceral fat area and the subcutaneous fat area and estimates the visceral fat area and the subcutaneous fat area using the extracted characteristic coefficients of the contour.

Specifically, the smartphone 1 extracts the characteristic coefficients of the contour of the cross-section after correcting the contour of the cross-section, for example. Methods of extracting the characteristics of a curved shape include a method of calculating a curvature function. In the present embodiment, however, a method using Fourier analysis is described. By subjecting one circumference of the contour of the cross-section to Fourier analysis, the controller 10 can seek the Fourier coefficients. As is well known, the Fourier coefficients of different orders that are sought when the curve is subjected to Fourier analysis are used to indicate the characteristics of the shape. The orders of Fourier coefficients that are extracted as characteristic coefficients are determined when deriving estimation formulas, which are described below in detail. In the present embodiment, the Fourier coefficients Sa₁, Sa₂, Sa₃, Sa₄ that affect the visceral fat area are extracted as characteristic coefficients of the visceral fat. Similarly, the Fourier coefficients Sb₁, Sb₂, Sb₃, Sb₄ that affect the subcutaneous fat area are extracted as characteristic coefficients of the subcutaneous fat. If the independent variables of each estimation formula are taken to be the principal components when the estimation formula is derived, then the principal components may be extracted as the characteristic coefficients.

The smartphone 1 estimates the user's visceral fat area and subcutaneous fat area by substituting the extracted characteristic coefficients Sa₁ to Sa₄ and Sb₁ to Sb₄ into the visceral fat area estimation formula and the subcutaneous fat area estimation formula calculated in advance. Examples of the visceral fat area estimation formula and the subcutaneous fat area estimation formula are illustrated in Equations 1 and 2.

A=−483.8+46.2×Sa₁−13.6×Sa₂+36.8×Sa₃+43.2×Sa₄ [Equation 1]

B=−280.0+41.6×Sb₁−24.9×Sb₂+16.6×Sb₃−40.0×Sb₄ [Equation 2]

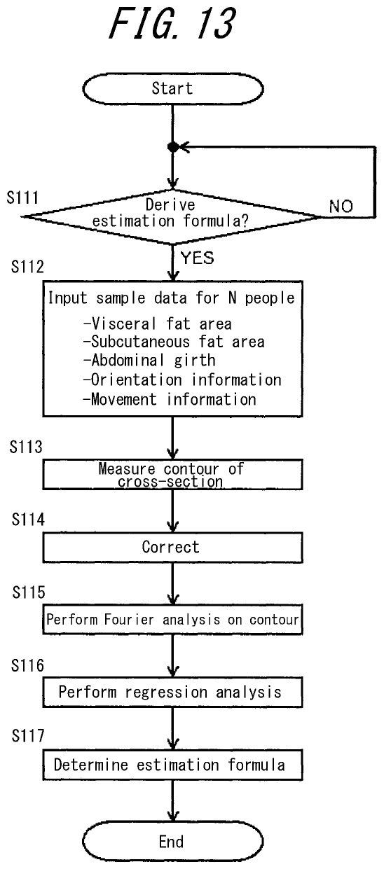

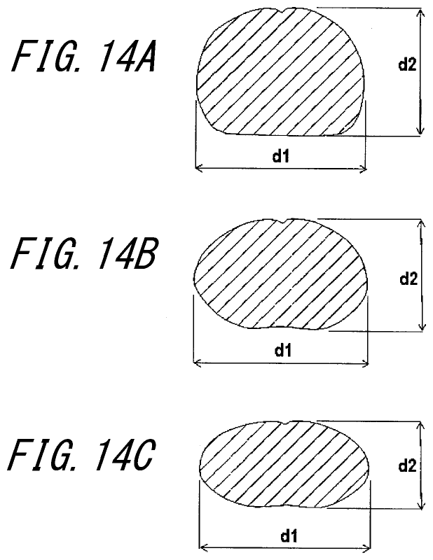

The method of deriving the visceral fat area estimation formula and the subcutaneous fat area estimation formula is now described. FIG. 13 is a flowchart for deriving the visceral fat area estimation formula and the subcutaneous fat area estimation formula. The procedure for deriving Equation 1 and Equation 2 is described with reference to FIG. 13. These estimation formulas need not be derived on the smartphone 1 and may be calculated in advance on another apparatus, such as a computer. The derived estimation formulas are read into the application in advance. Therefore, the user need not derive or change the estimation formulas directly.

In step S111, the author derives an estimation formula. In step S112, the author inputs sample data, acquired in advance, for a predetermined number of people into the computer. The sample data is acquired from a predetermined number of sample subjects. The sample data for one subject at least includes the visceral fat area and subcutaneous fat area obtained by CT, the abdominal girth measured by a tape measure or other instrument, orientation information acquired by the smartphone 1, and movement information. To improve accuracy of the estimation formulas, the predetermined number of sample subjects may be a statistically significant number and may be a group having a similar distribution to the visceral fat distribution of subjects for metabolic syndrome (MS) diagnosis.

Next, the computer calculates the contour of the cross-section from the inputted abdominal girth, orientation information, and movement information (step S113). Furthermore, the computer corrects the calculated contour of the cross-section (step S114).

Next, the computer performs Fourier analysis on the curve of the calculated, corrected contour of the cross-section (step S115). By subjection of the curve of the contour of the cross-section to Fourier analysis, a plurality of Fourier coefficients can be sought. As is well known, the Fourier coefficients of different orders that are obtained when the curve is subjected to Fourier analysis are used to represent the characteristics of the shape. In the present embodiment, the sample data for a predetermined number of people is subjected to Fourier analysis to seek the x-axis, y-axis, and 1^{st} to k^{th} order Fourier coefficients (where k is any integer). Furthermore, the Fourier coefficients may be subjected to well-known principal component analysis to reduce the number of dimensions. As the analysis method for principal component analysis, a common component may be sought for multivariate data (in the present embodiment, a plurality of Fourier coefficients), and a type of composite variable (principal component) may be derived. The characteristics of the curve can thus be represented with even fewer variables.

Next, regression analysis is performed with the plurality of Fourier coefficients (or principal components) sought in step S115 and the visceral fat area inputted in advance (step S116). Regression analysis refers to a statistical method for examining and clarifying the relationship between a numerical value representing a result and a numerical value representing a cause. With the Fourier coefficients (or principal components) as independent variables and the visceral fat area obtained by CT as a dependent variable, regression analysis is performed using the data of a predetermined number of sample subjects to derive the visceral fat area estimation formula (step S117). Similar calculations are made for the subcutaneous fat area to derive the subcutaneous fat area estimation formula.

Equation 1 and Equation 2 above are examples of the estimation formulas derived in this way. The independent variables Sa₁, Sa₂, Sa₃, Sa₄, and Sb₁, Sb₂, Sb₃, Sb₄ in Equation 1 and Equation 2 are the characteristic coefficients for estimating the user's visceral fat area and subcutaneous fat area. A portion or all of the characteristic coefficients Sa₁ to Sa₄ of the visceral fat area estimation formula and the characteristic coefficients Sb₁ to Sb₄ of the subcutaneous fat area may be the same Fourier coefficients. In this way, the estimation formulas for visceral fat area and subcutaneous fat area can be derived by the above-described statistical means (such as principal component analysis and regression analysis).

The estimation formulas are derived in step S116 by performing regression analysis for the visceral fat area and the subcutaneous fat area. An estimation formula can also be derived with a similar method for the circumference of an abdominal cross-section. In other words, regression analysis is performed with the plurality of Fourier coefficients (or principal components) sought in step S115 and the abdominal girth inputted in advance. With the Fourier coefficients (or principal components) as independent variables and the abdominal girth measured by a tape measure or other instrument as the dependent variable, regression analysis can be performed using the data of a predetermined number of sample subjects to derive the estimation formula for the circumference of the abdominal cross-section.

The smartphone 1 according to the present embodiment can use the above-described method to easily measure the contour of the cross-section of the abdomen accurately. The smartphone 1 can therefore quickly estimate the visceral fat area and the subcutaneous fat area accurately.

Referring again to FIG. 12, the information related to the contour of the abdominal cross-section may, for example, include the vertical and horizontal length (width) and the aspect ratio. The vertical and horizontal length and the aspect ratio are, for example, estimated based on the calculated contour of the cross-section of the abdomen. The horizontal length of the abdominal cross-section is the width of the abdominal cross-section in a front view of the person. The horizontal length of the abdominal cross-section is the width of the abdominal cross-section in the x-axis direction in FIG. 9. The vertical length of the abdominal cross-section is the width of the abdominal cross-section in a side view of the person and is the width in the direction orthogonal to the horizontal width of the abdominal cross-section. The vertical length of the abdominal cross-section is the width of the abdominal cross-section in the y-axis direction in FIG. 9. The aspect ratio of the abdominal cross-section is the ratio of the vertical length to the horizontal length of the abdominal cross-section.

The classification of the contour of the abdominal cross-section may be stored in the smartphone 1 in advance. FIGS. 14A, 14B, and 14C are conceptual diagrams illustrating example classifications of the contour of the abdominal cross-section. The classifications of the contour of the abdominal cross-section illustrated in FIGS. 14A, 14B, and 14C are A visceral obesity, B subcutaneous fat, and C average. The user is classified into one of A to C by the aspect ratio (d2/d1 in FIG. 14) of the measured contour of the abdominal cross-section. For example, an aspect ratio of 0.8 or more is classified as A visceral obesity, 0.6 or more to less than 0.8 as B subcutaneous fat, and less than 0.6 as C average. In this case, a [classification] step is added after step S105 in the flowchart in FIG. 6. The user can receive the result of classification and/or advice in accordance with the classification. The classification may be stored in the storage 9 along with the vertical and horizontal lengths of the abdominal cross-section and the aspect ratio.

The user may, for example, measure the contour of the abdominal cross-section with the smartphone 1 regularly and continuously. The contour of the abdominal cross-section may, for example, be measured every day, once a week, or once a month. The contour of the abdominal cross-section may be measured in the same time slot of the day. For example, the contour of the abdominal cross-section may be measured before a 7:00 am meal. Data can be acquired under the same conditions more easily when the contour of the abdominal cross-section is measured in the same time slot.

Referring again to FIG. 12, information related to food or drink and information related to physical activity are stored in the storage 9 in association with the time, for example.

The information related to food or drink may include a food menu, the user's calories consumed, beverages, health food, and medicine. The name of the food or drink and the amount thereof, for example, are stored in the storage 9 as the information related to food or drink.

The information related to physical activity may include the calories burned by the user and the hours of sleep.

The smartphone 1 can acquire the information related to food or drink and the information related to physical activity by the above-described methods, for example.

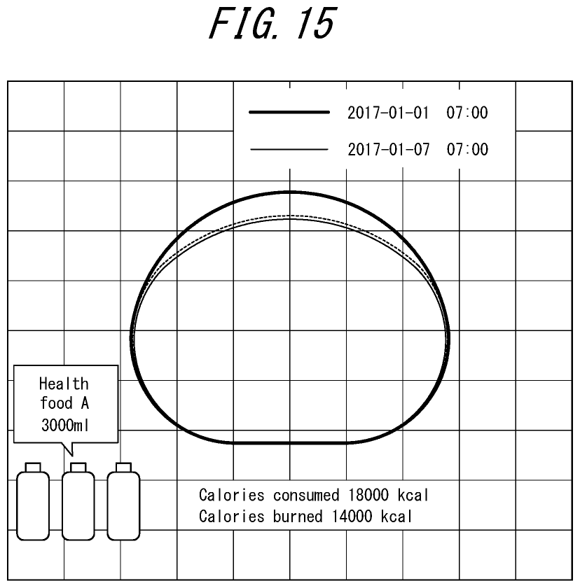

FIG. 15 illustrates an example display by the smartphone 1. The smartphone 1 can display the abdominal cross-section stored in the storage 9 on the display 2A. The smartphone 1 may display two contours measured at different times in overlap. In other words, the smartphone 1 may display a first contour measured at a first time and a second contour measured at a second time in overlap. In the example in FIG. 15, the first contour was generated at a first time of 7:00 am on Jan. 1, 2017, and the second contour was generated at a second time of 7:00 am on Jan. 7, 2017. This display of two contours in overlap allows the user to understand the change over time in the contour of the abdominal cross-section.

The two contours displayed in overlap can, for example, be determined automatically by the controller 10. For example, when the controller 10 measures the contour of the abdominal cross-section, the controller 10 may determine to display the measured contour in overlap with a contour measured at a predetermined earlier time (such as the previous day, week, or month). The two contours displayed in overlap may, for example, be determined based on user selection. In this case, the user can learn the change in the contour of the abdominal cross-section between two desired time periods (dates and times).

The two contours may be displayed in overlap with a predetermined position of the contours as a reference point. For example, the two contours may be displayed in overlap with the center of the back as a reference point, so that the centers of the back coincide. The center of the back is the position of the center at the back (rear) side of the user in the contour of the abdominal cross-section, such as the central portion along the horizontal length (width) at the back side. When the two contours are displayed in overlap with the center of the back as a reference point, the user can easily understand the change in the shape of the abdominal cross-section at the abdominal side.

The two contours may, for example, be displayed in overlap with the central portion of each contour as a reference point, so that the central portions coincide. The central portion of the contour of the abdominal cross-section is the intersection between the centers in a front view and a side view of the user, i.e. the intersection between the vertical and horizontal widths of the contour of the abdominal cross-section. When the two contours are displayed in overlap with the central portion of the contour of the abdominal cross-section as a reference point, the user can easily understand the change in overall size in the shape of the abdominal cross-section.

The number of contours that the smartphone 1 displays in overlap is not necessarily two. The smartphone 1 may display three or more contours in overlap. In this case as well, the user can understand the change over time in the contour of the abdominal cross-section.

In addition to two cross-sections, the smartphone 1 may display a predetermined virtual contour of an abdominal cross-section in overlap, as illustrated by the dashed line in FIG. 15, for example. The virtual contour illustrated by the dashed line in FIG. 15 has an aspect ratio of 0.78. The virtual contour may, for example, be an index such as a state of health. The virtual contour can, for example, be an index indicating the possibility of the user having a fatty liver. When a virtual contour is displayed in overlap, the user can easily compare and understand the contour of the user's abdominal cross-section and a contour serving as a predetermined index.

In addition to the two cross-sections, the smartphone 1 may display information related to food or drink and/or information related to physical activity between the times at which the two cross-sections were measured (between the first time and the second time), as illustrated in FIG. 15. The total number of calories consumed and the total number of calories burned by the user between the times at which the two cross-sections were measured are displayed in the example in FIG. 15. The name and amount of the health food consumed by the user between the times at which the two cross-sections were measured are displayed in the example in FIG. 15. The information related to food or drink and information related to physical activity that are displayed are not limited to the example in FIG. 15. For example, a portion or all of the data stored in the storage 9, an example of which is illustrated in FIG. 12, may be displayed together with the two cross-sections. The display of the information related to food or drink and/or information related to physical activity between the times at which the two cross-sections were measured makes it easier for the user to guess the relationship that food or drink and/or physical activity has with the change in shape of the contour of the abdominal cross-section. For example, when information related to food or drink is displayed, the user can easily understand how the shape of the contour changed in response to certain eating habits.

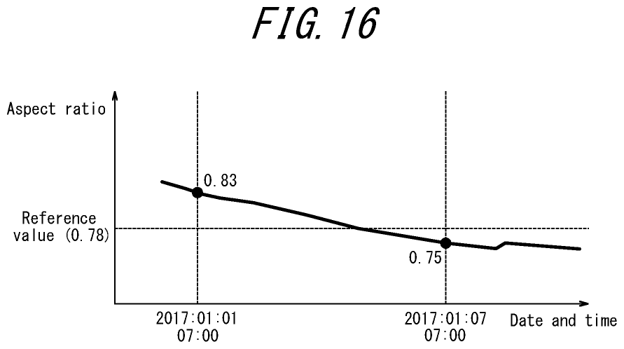

The smartphone 1 may display information related to the measured contour by another method. The smartphone 1 may, for example, display a graph of the change over time in the measured abdominal girth, horizontal width (horizontal length), and vertical width (vertical length). The smartphone 1 may, for example, display a graph of the change over time in the aspect ratio of the measured abdominal girth. FIG. 16 is an example of a graph illustrating the change in the aspect ratio. In the graph in FIG. 16, the vertical axis represents the aspect ratio, and the horizontal axis represents the time of measurement. By information related to the contour being depicted as a graph, the user can easily understand the change over time in the information related to the contour. The smartphone 1 may indicate a value (reference value: 0.78) serving as an index of the state of health on the graph, as in FIG. 16, for example. When the reference value is indicated on the graph, the user can easily compare the values related to the user's contour with the reference value. This reference value may have a similar meaning to that of the above-described index related to the virtual contour.

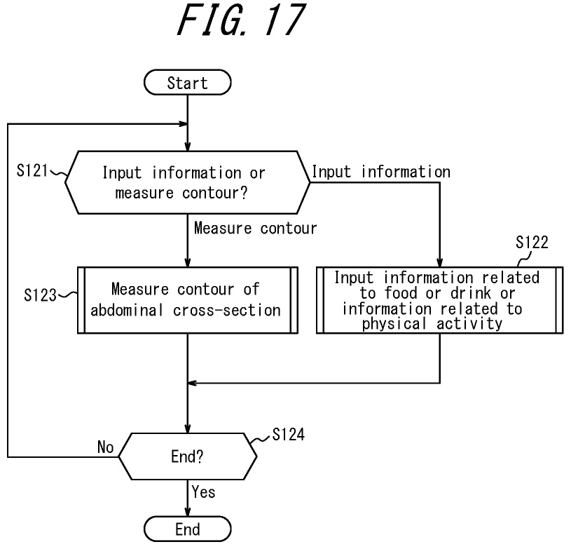

FIG. 17 is a flowchart of the entire processing executed by the smartphone 1 according to the present embodiment. In step S121, the smartphone 1 determines whether to input information or to measure a contour based on operation input from the user.

When the smartphone 1 determines in step S121 to input information, the smartphone 1 receives input of information related to food or drink or information related to physical activity based on user operation input in step S122. Details on the input of information related to food or drink or information related to physical activity are as described above. The input of information may, for example, include capturing an image of food or drink or receiving input of calories consumed.

After the smartphone 1 receives input of information in step S122, the smartphone 1 judges in step S124 whether the user has inputted an instruction to end processing. When the smartphone 1 judges that an instruction to end processing has been inputted, the smartphone 1 terminates the processing flow in FIG. 17. Conversely, when the smartphone 1 judges that an instruction to end processing has not been inputted (for example, when an instruction to continue processing has been inputted), the smartphone 1 proceeds to step S121.

When the smartphone 1 determines in step S121 to measure the contour, the smartphone 1 measures the contour of the abdominal cross-section in step S123. Details of step S123 are as described with reference to FIG. 6. The smartphone 1 may display the measured contour after measuring the contour.

After the smartphone 1 measures the contour in step S123, the smartphone 1 judges in step S124 whether the user has inputted an instruction to end processing. When the smartphone 1 judges that an instruction to end processing has been inputted, the smartphone 1 terminates the processing flow in FIG. 17. Conversely, when the smartphone 1 judges that an instruction to end processing has not been inputted (for example, when an instruction to continue processing has been inputted), the smartphone 1 proceeds to step S121.

### Second Embodiment

FIG. 18 is a block diagram illustrating the configuration of a smartphone 1 according to the second embodiment.

In the present embodiment, a timer 11 and a control unit 10A are included in a controller 10. The timer 11 is a device for obtaining movement information of the smartphone 1. The timer 11 receives an instruction for a timer operation from the control unit 10A and outputs a clock signal. The direction sensor 17 acquires orientation information multiple times in accordance with the clock signal outputted from the timer 11. The orientation information acquired in accordance with the clock signal is temporarily stored inside the smartphone 1 along with clock information. Clock information refers to information indicating the time at which the orientation information was acquired. For example, the clock information may be a record number indicating the order of acquisition when using a clock signal with a constant period. The clock information may also be the time of acquisition of the orientation information. In the present embodiment, the timer 11 is included in the controller 10. A timer circuit that is a functional component of the controller 10 may be used as the timer 11. The present disclosure is not limited to this example. As described above with reference to FIG. 4, the timer 11 may be provided externally to the controller 10.

The control unit 10A estimates the movement information of the smartphone 1 from the clock information. The movement information of the smartphone 1 is related to the movement amount of the smartphone 1. In the present embodiment, the movement information is the movement amount. The processor 10A calculates a contour of a cross-section of an object based on the movement information. The differences from the first embodiment are described below, with a description of common features being omitted.

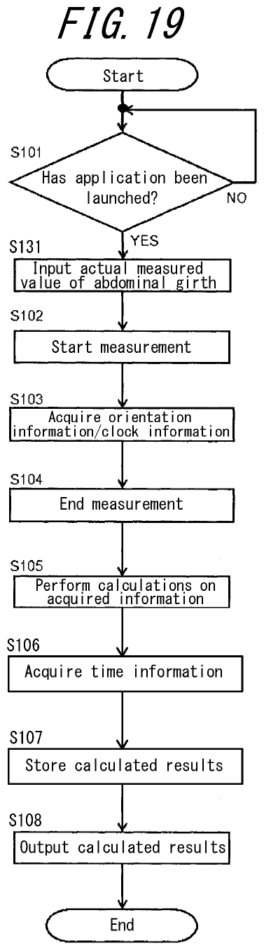

FIG. 19 is a flowchart for measurement of the contour of an abdominal cross-section according to the second embodiment.

In step S101, the user launches the measurement application 9Z for measuring the contour of a cross-section. After launching the measurement application 9Z, the user inputs the actual measured value of the abdominal girth, as measured in advance with a tape measure or other instrument, into the smartphone 1 (step S131). Alternatively, the smartphone 1 may read the actual measured value of the abdominal girth from user information stored in advance in the storage 9. The actual measured value of the abdominal girth need not be inputted before the start of measurement (step S102) and may instead be inputted after measurement is complete (step S104).

Next, measurement begins in step S102. At the start of measurement, the smartphone 1 is placed against the surface of the abdomen 60 at any position where the contour of an abdominal cross-section is to be measured. In the present embodiment, the contour of a cross-section at the height of the user's navel (the position indicated by A-A in FIG. 5) is measured. The measurement start position may be anywhere along the abdominal A-A position. To start measurement, the user performs a preset start action on the smartphone 1. In step S103, the user moves the smartphone 1 along the surface at the A-A position of the abdomen 60. The user moves the smartphone 1 at constant speed while keeping the smartphone 1 against the surface of the abdomen 60. A support tool that facilitates movement of the smartphone 1 may be employed so that the user can move the smartphone 1 at constant speed. A supporting sound may be outputted at constant speed from the smartphone 1 to guide the operation.

In step S103, the smartphone 1 acquires orientation information with the direction sensor 17 under pre-programmed conditions. The orientation information is acquired multiple times in accordance with the clock signal outputted from the timer 11. The orientation information acquired in accordance with the clock signal is stored in the smartphone 1 along with the clock information. This measurement is continuously made from the start of step S102 until the end of step S104.

The user moves the smartphone 1 around the abdomen 60 once or more at constant speed while keeping the smartphone 1 against the surface of the abdomen 60. Subsequently, the user performs a preset end action on the smartphone 1 and ends measurement (step S104). Alternatively, the smartphone 1 may end measurement automatically, without user operation, by recognizing a complete circumference when the orientation information acquired by the direction sensor 17 of the smartphone 1 matches the orientation information at the start of measurement. The smartphone 1 may also end measurement automatically, without user operation, by recognizing a complete circumference when the orientation information acquired by the direction sensor 17 of the smartphone 1 changes by 360° from the orientation information at the start of measurement. In the case of automatic recognition, the user need not perform the end action, thereby simplifying measurement.

In step S105, the control unit 10A estimates the movement amount, which is the movement information of the smartphone 1, by the actual measured value of the user's abdominal girth and the clock information acquired in step S103. The circumferential movement amount of the smartphone 1 once around the user's abdominal girth is equivalent to the actual measured value of the abdominal girth inputted in step S111, and the smartphone 1 is considered to move at a constant speed. Therefore, the movement amount can be calculated as the movement information of the smartphone 1. The processor 10A calculates the contour of a cross-section of the object based on the acquired orientation information and the calculated movement information.

In step S106, the smartphone acquires time information indicating the time of measurement.

In step S107, the smartphone 1 associates the result of calculations in step S105 with the time information acquired in step S106 and stores the associated result and time information in the storage 9.

In step S108, the smartphone 1 may output the results of the calculations in step S105. Once output of the results of calculating the contour and girth of the cross-section of the abdomen is complete, the smartphone 1 ends the processing flow. The other operations not described in detail in the flowchart of the present embodiment conform to the operations in FIG. 6.

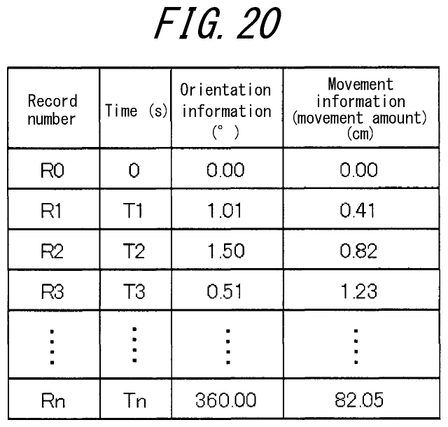

FIG. 20 is an example record formed by acquired information according to the second embodiment.

The record number at the start of measurement is R0, and the record number at the end of measurement is Rn. In each record, orientation information and movement information corresponding to time are stored as a pair. The movement information is the movement amount estimated from the record number (or the time), which is clock information. The actual measured value of the user's abdominal girth is stored as the movement information of record number Rn. The time intervals between records are equal intervals, and the smartphone 1 is considered to move at a constant speed. Therefore, the interval between each movement amount, which is movement information, is also an equal interval. Records acquired in this way are displayed as a diagram indicating the contour of a cross-section.

The contour of a cross-section of the object can be calculated by plotting the xy coordinates of the acquired records R0 to Rn in order in accordance with orientation and movement amount. In the present embodiment, each plotted point is at an equal interval in the calculated contour of a cross-section illustrated in FIG. 9. When movement of the smartphone 1 is at a constant speed at the time of measurement, the calculated contour of a cross-section has a nearly symmetrical shape about the y-axis. When movement of the smartphone 1 is not at a constant speed at the time of measurement, the calculated contour of a cross-section has a non-symmetrical, irregular shape about the y-axis. When the shape of the calculated contour of a cross-section is highly non-symmetrical, a message encouraging the user to measure again at constant speed may be displayed on the smartphone 1. The judgment of the magnitude of non-symmetry may be made on the basis of the difference in the number of plotted points in the two regions separated by the y-axis in FIG. 9. For example, when the difference in the number of plotted points is other than ±10%, the contour of the cross-section may be determined to be highly non-symmetrical. The method for determining the degree of non-symmetry is not limited to this example. For example, areas surrounded by the contour of the cross-section may be calculated and compared to determine the degree of non-symmetry. The standard for judgment may be set as necessary.

In the present embodiment, use of the timer 11 as the device for obtaining movement information of the electronic device allows the movement information to be acquired without use of the second sensor. Therefore, the number of components can be further reduced in the smartphone 1 of the present embodiment. Furthermore, the smartphone 1 of the present embodiment can reduce the measurement error attributable to the accuracy of the second sensor.

The method by which the smartphone 1 according to the present embodiment acquires information related to food or drink and information related to physical activity may be similar to the first embodiment. The method by which the smartphone 1 according to the present embodiment displays the contour of the abdominal cross-section may also be similar to the first embodiment. The user can more easily understand the change over time in the contour of the abdominal cross-section with the smartphone 1 according to the present embodiment as well.

### Third Embodiment

In the third embodiment, the contour of an abdominal cross-section is estimated from a portion of a calculated contour of a cross-section. Furthermore, an image of the contour of the abdominal cross-section from the estimated value is displayed on the smartphone 1. The smartphone 1 of the present embodiment may have the same configuration as the block diagram of FIG. 18 in the second embodiment. The differences from the first and second embodiments are described below, with a description of common features being omitted.

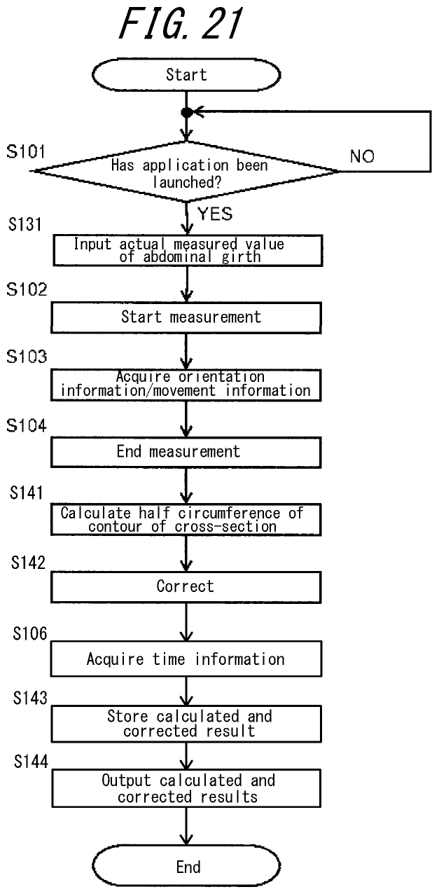

FIG. 21 is a flowchart illustrating an example of processing up to display of a contour image of an abdominal cross-section according to the third embodiment. In the present embodiment, as an example of calculating at least a partial contour of an abdominal cross-section, the case of calculating the half-circumferential contour from the position of the navel is described.

In step S101, the user launches the measurement application 9Z for measuring the contour of a cross-section. After launching the measurement application 9Z, the user inputs the actual measured value of the abdominal girth, as measured in advance with a tape measure or other instrument, into the smartphone 1 (step S131). Alternatively, the actual measured value of the abdominal girth may be read from user information stored in advance in the storage 9 of the smartphone 1. Step S131 need not be performed before the start of measurement and may instead be performed after measurement in step S104 is complete.

Next, measurement begins in step S102. At the start of measurement, the smartphone 1 is placed against the surface of the abdomen 60 at the position of the navel. The measurement start position may be selected in accordance with the portion of the abdominal cross-section for which the contour is to be calculated. By determining the measurement start position in advance, the range of the calculated contour does not change from user to user, reducing the error in the below-described characteristic coefficients of the contour. In the present embodiment, the position of the navel is the measurement start position. For example, the side face 1C1 of the smartphone 1 is matched to the position of the navel, and measurement is started. The user starts measurement by performing a preset start action on the smartphone 1.

In step S103, the user moves the smartphone 1 along the surface at the

A-A position of the abdomen 60. The user moves the smartphone 1 at constant speed while keeping the smartphone 1 against the surface of the abdomen 60.

In step S103, the smartphone 1 acquires the angular velocity (°/s), which is orientation information, with the angular velocity sensor 18 under pre-programmed conditions. The orientation information is acquired multiple times in accordance with the clock signal outputted from the timer 11. The orientation information acquired in accordance with the clock signal is stored in the smartphone 1 along with acquired time information. This measurement is continuously made from the start of step S102 until the end of step S104.

The user moves the smartphone 1 around the abdomen 60 over half or more of the circumference at constant speed while keeping the smartphone 1 against the surface of the abdomen 60. In the present embodiment, half of the circumference refers to moving from the navel to the center of the back. Accordingly, the smartphone 1 may include means for notifying the user of the half circumference.

After moving the smartphone 1 over half or more of the circumference, the user performs a preset end action on the smartphone 1 and ends measurement (step S104). Alternatively, if the below-described step S141 is executed simultaneously, the smartphone 1 may end measurement automatically by recognizing nearly half of the circumference when the orientation of the smartphone 1 changes 180° from the start of measurement. With such automatic recognition, the user need not perform the end action, which simplifies measurement.

After the end of measurement or during measurement, the processor 10A calculates the half-circumferential contour of the abdominal cross-section (step S141). The control unit 10A calculates the orientation of the smartphone 1 by integrating the angular velocity, acquired in step S103, once.

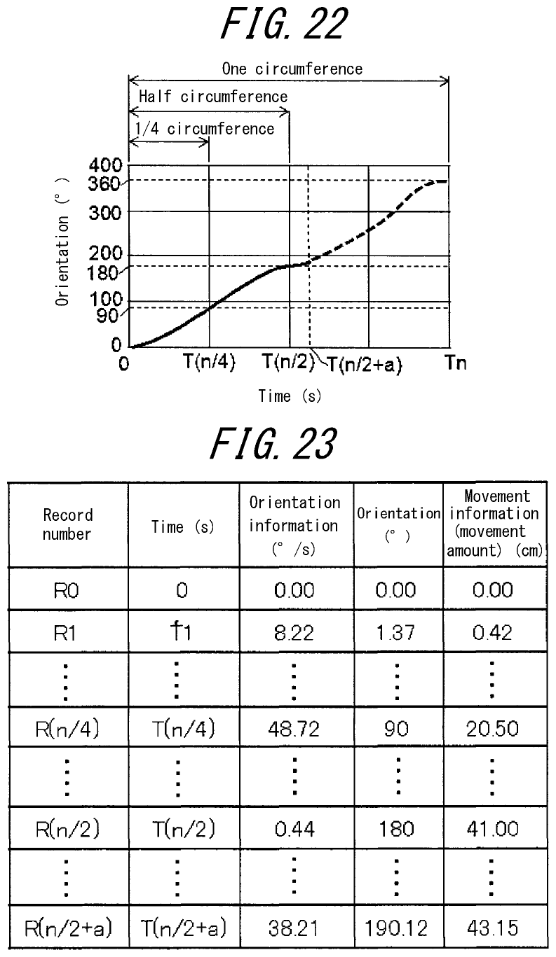

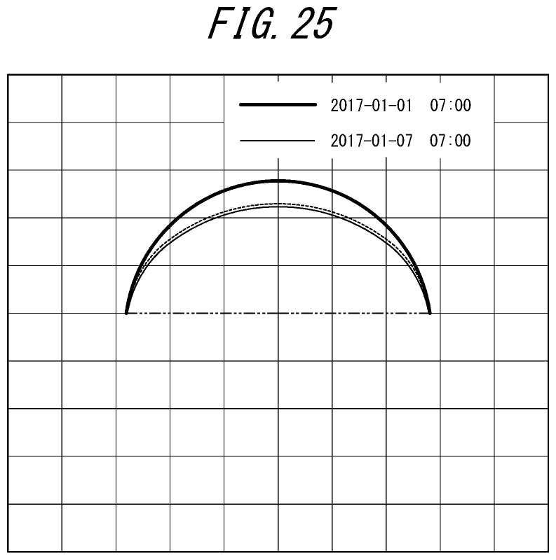

FIG. 22 illustrates an example orientation of the smartphone 1 according to the third embodiment. With reference to FIG. 22, the method of extracting information on the half circumference from the acquired orientation information is described. The horizontal axis represents time. The measurement start time is 0 s, and the measurement end time is T(n/2+a) s. Here, n represents 360° (one circumference), and a represents the angle yielded by subtracting 180° (half circumference) from the orientation at the measurement end time. The vertical axis represents the orientation of the smartphone 1. The solid line represents acquired information, whereas the dotted line is an imaginary line of non-acquired information for the full circumference. The flat portion of the curve in FIG. 25 where the orientation is near 180° is estimated to be information on the back. The smartphone 1 judges that the center of the back has been passed at the center of this flat portion and detects the half circumference. In other words, the smartphone 1 extracts the time T(n/2) s after 0 s in FIG. 25 as information on the half circumference. This method of extracting information on the half circumference is only an example. For example, when the flat portion is at a position shifted from 180°, the smartphone 1 may normalize the flat portion to 180°. The smartphone 1 may perform normalization by setting the position where the orientation is −180° from the flat portion as the starting point. Rather than the center of the flat portion, the smartphone 1 may judge that the position where the inclination of the curve is smallest near the orientation of 180° is the center of the back.

FIG. 23 is an example record formed by acquired and normalized information according to the third embodiment. The extracted starting point of the half circumference of the contour (in the present embodiment, the position of the navel) is set to record number R0, the ending point of the half circumference (in the present embodiment, the record where the orientation is 180° at the center of the back) is set to record R(n/2), and the last acquired information is set to record R(n/2+a). In each record, orientation information and movement information are stored as a pair. The movement information is the movement amount estimated from the record number (or the time), which is clock information. In the present embodiment, records for an orientation of 0° to 180° are extracted as information on the half circumference. Half of the actual measured value of the user's abdominal girth is stored as the movement information of record number R(n/2). The time intervals between records are equal intervals, and the smartphone 1 is considered to move at a constant speed. Therefore, the interval between each movement amount, which is movement information, is also an equal interval. Records acquired in this way are displayed as a diagram indicating the half-circumferential contour of a cross-section. The smartphone 1 can calculate the half circumference of the contour of the object by plotting the xy coordinates of the acquired records R0 to R(n/2) in order in accordance with orientation and movement amount. Step S141 may be executed in parallel with step S103.

In step S142, the smartphone 1 corrects the results of the calculations in step S141. The orientation of the contour and the position of the contour may be corrected based on an inverted closed curve yielded by folding the calculated half circumference of the contour of the cross-section over an axis of symmetry defined by a line connecting the starting point (the position of the navel in the present embodiment) and the ending point (the center of the back in the present embodiment). To correct the orientation of the contour, the inverted closed curve may be rotated so that the axis of symmetry of the inverted closed curve (the line connecting the navel and the center of the back) faces a predetermined direction. To correct the position of the contour, the inverted closed curve may be moved so that the center point of the inverted closed curve coincides with the origin of the coordinate system. The orientation and position may be corrected by a known method.

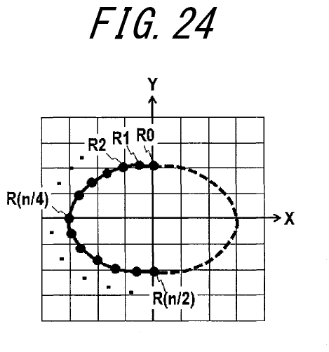

FIG. 24 illustrates a calculated and corrected contour of a cross-section according to the third embodiment. The solid line in the graph is the calculated half-circumferential contour of the cross-section, and the dotted line is the imaginary line when the calculated half-circumferential contour of the cross-section is rotated about the axis of symmetry. The black dots are plots of the acquired records on the xy coordinates. The controller 10 can, in this way, derive the contour of an abdominal cross-section.

In step S106, the smartphone acquires time information indicating the time of measurement.

In step S143, the smartphone 1 associates the results of calculations and correction in steps S141 and S142 with the time information acquired in step S106 and stores the associated results and time information in the storage 9.

In step S144, the smartphone 1 may output the results of calculations and correction in steps S141 and S142. Once output of the results of calculating the contour and girth of the cross-section of the abdomen is complete, the smartphone 1 ends the flow.

The method by which the smartphone 1 according to the present embodiment acquires information related to food or drink and information related to physical activity may be similar to the first embodiment. The method by which the smartphone 1 according to the present embodiment displays the contour of the abdominal cross-section may also be similar to the first embodiment. The user can more easily understand the change over time in the contour of the abdominal cross-section with the smartphone 1 according to the present embodiment as well.

The contour of a person's abdominal cross-section is nearly symmetrical. Therefore, by simply calculating at least the half-circumferential contour of a cross-section, the smartphone 1 of the present embodiment can estimate the contour of the abdominal cross-section. As a result, it suffices for the user to move the smartphone 1 around at least half of the abdomen, thereby shortening the measurement time. Furthermore, the smartphone 1 no longer needs to be switched between hands during measurement, making it easier to move the smartphone 1 at a constant speed and improving measurement accuracy.

Instead of calculating the contour of the abdominal cross-section from the half circumference, the smartphone 1 may calculate the contour of the abdominal cross-section from a ¼ circumference. For example, the case of calculating the contour of the abdominal cross-section based on the ¼ circumference from the navel to the side is described. The process is similar to the above-described process of FIG. 21, replacing the half circumference with a ¼ circumference. To calculate the ¼ circumference in step S141, a substantially ¼ circumference may be judged when the orientation of the smartphone 1 has changed 90° from the start of measurement, for example. It is judged that the ¼ circumference point has been passed at an orientation of 90° in the graph of the orientation of the smartphone 1 in FIG. 22, and a ¼ circumference is detected. In other words, the portion from 0 s to T(n/4) s in FIG. 25 is extracted as information on the ¼ circumference. The records for the orientation from 0° to 90° in FIG. 23 are extracted as information on the ¼ circumference. In the example records of FIG. 23, the ending point of the ¼ circumference is record R(n/4). One quarter of the actual measured value of the user's abdominal girth is stored as the movement information of record number R(n/4). The smartphone 1 moves at a constant speed. The interval between each movement amount, which is movement information, is therefore also an equal interval. The ¼ circumference of the contour of the cross-section of the object can be calculated by plotting the records R0 to R(n/4) acquired in this way in order, in accordance with orientation and movement amount. The orientation and position of the contour can easily be corrected in step S142 based on the inverted closed curve yielded by folding the calculated ¼ circumference of the contour over the y-axis and x-axis of the coordinate system as axes of symmetry. In this case, the estimation formula in the above-described FIG. 13 may be derived by changing the half circumference to a ¼ circumference. This method of extracting the ¼ circumference is only an example. When the time at which the orientation becomes 180° is T(n/2), for example, the record at half of that time may be extracted as the ¼ circumference.

In this case, the smartphone 1 can estimate the contour of the abdominal cross-section by calculating at least a ¼ circumference of the contour of the cross-section. As a result, it suffices for the user to move the smartphone 1 around at least ¼ of the abdomen, thereby shortening the measurement time. Furthermore, the smartphone 1 no longer needs to be circled around to the back during measurement, making it easier to move the smartphone 1 at a constant speed and further improving measurement accuracy.

The ¼ circumference from the navel to the side has been illustrated in the present embodiment, but the present disclosure is not limited to this example. The contour of the abdominal cross-section can be estimated by calculating the ¼ circumference from near the side to the back.

When calculating the contour of the abdominal cross-section based on the ¼ circumference from the navel to the side, the smartphone 1 need not necessarily calculate the contour of the entire abdominal cross-section. The smartphone 1 may, for example, calculate only the front side of the user's abdominal cross-section. In this case, the smartphone 1 can perform corrections based on the inverted curve yielded by folding the calculated ¼ circumference of the contour over the y-axis of the coordinate system as an axis of symmetry. The smartphone 1 may display only the contour of the front side of the abdominal cross-section in this case, as illustrated in FIG. 25, for example. The front side tends to change more than the back side in the abdominal cross-section. The user can therefore understand the change in the contour of the abdominal cross-section even when only the front side is displayed.

In the present embodiment, the smartphone 1 can estimate the contour of the abdominal cross-section and the circumference of the abdominal cross-section even when the orientation information and the movement information are acquired for less than the half circumference of the abdomen. For example, the smartphone 1 may estimate the contour of the abdominal cross-section and the circumference of the abdominal cross-section based on contour information from the navel position to the 135° position (⅜ of the circumference).

Next, a system according to an embodiment of the present disclosure is described in detail with reference to the drawings.

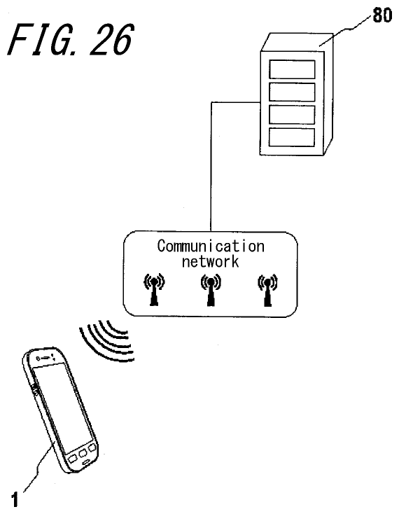

The system according to the present embodiment in FIG. 26 includes a server 80, a smartphone 1, and a communication network. As illustrated in FIG. 26, the smartphone 1 transmits the calculation result of the measured contour of the cross-section to the server 80 over a communication network. The smartphone 1 may also transmit acquired information related to food or drink and information related to physical activity to the server 80. The server 80 transmits data of a display image with two contours in overlap to the smartphone 1. The smartphone 1 can display the display image and the like transmitted from the server 80 on the display 2A. In this case, the display image is generated on the server 80. The burden of calculation on the controller 10 of the user's smartphone 1 can therefore be reduced, allowing the smartphone 1 to be reduced in size and simplified. A configuration may also be adopted to transmit the acquired orientation information, movement information, and abdominal girth to the server 80. In this case, the server 80 calculates the contour of the cross-section. The burden of calculation on the controller 10 of the user's smartphone 1 can therefore be further reduced. The processing speed for calculation also improves.

The server 80 may store at least one of the following pieces of data: a first time at which a first contour was measured; a second time at which a second contour was measured; the type, amount, and calories of food or drink consumed by the user between the first time and the second time; and the user's amount of exercise, calories burned, and hours of sleep. The server 80 may transmit data stored during a predetermined time period to the smartphone 1 in response to a request from the smartphone 1. Based on the data transmitted from the server 80, the smartphone 1 may display the first and second contours along with at least one of the type, amount, and calories of food or drink consumed by the user between the first time and the second time and the user's amount of exercise, calories burned, and hours of sleep.

As the system according to the present embodiment, a configuration in which the smartphone 1 and the server 80 are connected over a communication network has been illustrated. The system of the present disclosure is not, however, limited to this configuration. It suffices for the system to include a measuring instrument that is moved along the surface of an object, a first sensor configured to acquire orientation information of the measuring instrument, a device configured to obtain movement information of the measuring instrument, and a controller configured to calculate a contour of a cross-section of the object. These functional units may be connected by a communication interface.

Characteristic embodiments have been described for a complete and clear disclosure. The appended claims, however, are not limited to the above embodiments and are to be understood as encompassing all of the possible modifications and alternate configurations that a person of ordinary skill in the art could make within the scope of the fundamental features indicated in the present disclosure.

For example, the case of the electronic device being the smartphone 1 has been described in the above embodiments, but the electronic device of the present disclosure is not limited to the smartphone 1 and simply needs to include the first sensor, the device, and the controller. Furthermore, the first sensor, the device, and the controller need not be provided inside the electronic device and may be separate, individual components.

In the above embodiments, the case of measuring the contour of a cross-section of the abdomen has been described, but the contour of the torso, chest, thigh, or the like may also be measured. Besides a human abdomen, the contour of an animal abdomen, chest, torso, leg, or the like may also be measured. Such contours measured at different times may be displayed in overlap. A plurality of contours measured at different times may be displayed side-by-side for comparison.

In the above embodiments, the case of using the direction sensor 17 and the angular velocity sensor 18 as the first sensor has been described, but the first sensor may be any other component that can acquire orientation information of the electronic device. For example, an inclination sensor may be used as the first sensor.

The case of using the acceleration sensor 16 or the electronic tape measure 71 as the second sensor has been described, but the second sensor may be any other component that can acquire movement information of the electronic device. For example, an electronic roller distance meter that acquires movement information by detecting the number of revolutions of a wheel may be used as the second sensor.

In the above embodiments, examples of measuring the contour of the cross-section over one circumference, a half circumference, and a ¼ circumference have been described, but other lengths are possible. For example, the contour of the cross-section may be measured around the circumference twice, and the data may be averaged to allow highly accurate measurement with less variation.

Much of the subject matter of the present disclosure is described as a series of operations executed by a computer system and other hardware that can execute program instructions. Examples of the computer system and other hardware include a general-purpose computer, a personal computer (PC), a dedicated computer, a workstation, a personal communications system (PCS), a mobile (cellular) phone, a mobile phone with a data processing function, an RFID receiver, a game device, an electronic notepad, a laptop computer, a GPS receiver, and other programmable data processing apparatuses. It should be noted that in each embodiment, various operations are executed by a dedicated circuit (for example, individual logical gates interconnected in order to execute a particular function) implementing program instructions (software), or by a logical block, program module, or the like executed by one or more processors. The one or more processors that execute a logical block, program module, or the like include, for example, one or more of a microprocessor, CPU, Application Specific Integrated Circuit (ASIC), Digital Signal Processor (DSP), Programmable Logic Device (PLD), Field Programmable Gate Array (FPGA), processor, controller, microcontroller, microprocessor, electronic device, other apparatus designed to be capable of executing the functions disclosed here, and/or a combination of any of the above. The embodiments disclosed here are, for example, implemented by hardware, software, firmware, middleware, microcode, or a combination of any of these. The instructions may be program code or a code segment for executing the necessary tasks. The instructions may be stored on a machine-readable, non-transitory storage medium or other medium. The code segment may indicate a combination of any of the following: procedures, functions, subprograms, programs, routines, subroutines, modules, software packages, classes, instructions, data structures, or program statements. The code segment transmits and/or receives information, data arguments, variables, or memory content to or from another code segment or hardware circuit and thereby connects to the other code segment or hardware circuit.

The network used here may, unless indicated otherwise, be the Internet, an ad hoc network, a local area network (LAN), a wide area network (WAN), a metropolitan area network (MAN), a cellular network, a wireless wide area network (WWAN), a wireless personal area network (WPAN), a public switched telephone network (PSTN), a terrestrial wireless network, another network, or a combination of any of these. The constituent elements of a wireless network for example include an access point (such as a Wi-Fi access point), a femtocell, or the like. Furthermore, a wireless communication device can connect to a wireless network that uses Wi-Fi, Bluetooth®, cellular communication technology (such as code division multiple access (CDMA), time division multiple access (TDMA), frequency division multiple access (FDMA), orthogonal frequency division multiple access (OFDMA), or single-carrier frequency division multiple access (SC-FDMA)), or other wireless techniques and/or technical standards. One or more techniques may be adopted for the networks. Such techniques include, for example, Universal Mobile Telecommunications System (UTMS), Long Term Evolution (LTE), Evolution-Data Optimized or Evolution-Data Only (EV-DO), Global System for Mobile Communications (GSM®), Worldwide Interoperability for Microwave Access (WiMAX), Code Division Multiple Access-2000 (CDMA-2000), or Time Division Synchronous Code Division Multiple Access (TD-SCDMA).

The circuit configuration of the communication interface or other such components provides functionality by using a variety of wireless communication networks, such as WWAN, WLAN, and WPAN. The WWAN may be a network such as a CDMA network, a TDMA network, an FDMA network, an OFDMA network, or a SC-FDMA network. The CDMA network implements one or more Radio Access Technologies (RAT), such as CDMA2000 and Wideband-CDMA (W-CDMA). CDMA2000 includes the IS-95, IS-2000, and IS-856 standards. The TDMA network can implement GSM®, Digital Advanced Phone System (D-AMPS), or another RAT. GSM® and W-CDMA are listed in documents issued by the consortium known as 3^{rd} Generation Partnership Project (3GPP). CDMA2000 is listed in documents issued by the consortium known as 3^{rd} Generation Partnership Project 2 (3GPP2). The WLAN may be an IEEE802.11x network. The WPAN may be a Bluetooth® network, an IEEE802.15x network, or other type of network. CDMA may be implemented as a wireless technique such as Universal Terrestrial Radio Access (UTRA) or CDMA2000. TDMA may be implemented by a wireless technique such as GSM®/General Packet Radio Service (GPRS)/Enhanced Data Rates for GSM® Evolution (EDGE). OFDMA may be implemented by wireless techniques such as Institute of Electrical and Electronics Engineers (IEEE) 802.11 (Wi-Fi), IEEE802.16 (WiMAX), IEEE802.20, or Evolved UTRA (E-UTRA). These techniques may be used in a combination of any of WWAN, WLAN, and/or WPAN. These techniques may also be implemented in order to use an Ultra Mobile Broadband (UMB) network, a High Rate Packet Data (HRPD) network, a CDMA20001× network, GSM®, Long Term Evolution (LTE), or the like.

The storage 9 used here may also be configured by a computer-readable, tangible carrier (medium) in the categories of solid-state memory, magnetic disks, and optical discs. Data structures and an appropriate set of computer instructions, such as program modules, for causing a processor to execute the techniques disclosed herein are stored on these media. Examples of computer-readable media include an electrical connection with one or more wires, a magnetic disk storage medium, a magnetic cassette, a magnetic tape, or other magnetic or optical storage medium (such as a Compact Disc (CD), laser Disc®, DVD®, Floppy® disk, and Blu-ray® Disc (laser disc and floppy are registered trademarks in Japan, other countries, or both)), portable computer disk, random access memory (RAM), read-only memory (ROM), rewritable programmable ROM such as EPROM, EEPROM, or flash memory, another tangible storage medium that can store information, or a combination of any of these. The memory may be provided internally and/or externally to a processor or processing unit. As used in the present disclosure, the term “memory” refers to all types of long-term storage, short-term storage, volatile, non-volatile, or other memory. No limitation is placed on the particular type or number of memories, or on the type of medium for memory storage.

While the disclosed system has a variety of modules and/or units for implementing particular functions, these modules and units have only been indicated schematically in order to briefly illustrate the functionality thereof. It should be noted that no particular hardware and/or software is necessarily indicated. In this sense, it suffices for the modules, units, and other constituent elements to be hardware and/or software implemented so as to substantially execute the particular functions described herein. The various functions of different constituent elements may be implemented by combining or separating hardware and/or software in any way, and the functions may each be used individually or in some combination. An input/output (I/O) device or user interface including, but not limited to, a keyboard, display, touchscreen, or pointing device may be connected to the system directly or via an I/O controller. In this way, the various subject matter disclosed herein may be embodied in a variety of forms, and all such embodiments are included in the scope of the subject matter in the present disclosure.

### REFERENCE SIGNS LIST

1 Smartphone
1A, 71A Front face
1B Back face
1C1-4, 71C2 Side face
2, 72 Touchscreen display
2A Display
2B Touchscreen
3 Button
4 Illuminance sensor
5 Proximity sensor
6 Communication interface
7 Receiver
8 Microphone
9 Storage
9A Control program
9B Mail application
9C Browser application
9Z Measurement application
10 Controller
10A Control unit
11 Timer
12, 13 Camera
14 Connector
15 Motion sensor
16 Acceleration sensor
17 Direction sensor
18 Angular velocity sensor
19 Inclination sensor
20, 70 Housing
60 Abdomen
71 Electronic tape measure
73 Tape measure
74 Stopper
80 Server

1. An electronic device comprising:
a measurement unit configured to measure a contour of an abdomen; and
a controller configured to display the contour on a display,
the controller being further configured to display a first contour and a second contour that are measured at different times in overlap on the display.

2. The electronic device of claim 1, wherein the controller is configured to determine the first contour and the second contour based on user selection.

3. The electronic device of claim 1, wherein the controller is configured to display the first contour and the second contour in overlap using a central portion of a back in the first contour and the second contour as a reference point.

4. The electronic device of claim 1, wherein the controller is configured to display the first contour and the second contour in overlap using a center of the first contour and the second contour as a reference point.

5. The electronic device of claim 1, wherein the measurement unit comprises at least one of an acceleration sensor, a direction sensor, an angular velocity sensor, an inclination sensor, and a camera.

6. The electronic device of claim 1, wherein the controller is further configured to store in a storage at least one of a first time at which the first contour is measured; a second time at which the second contour is measured; a type, amount, and calories of food or drink consumed by a user between the first time and the second time; and an amount of exercise, calories burned, and hours of sleep of the user.

7. The electronic device of claim 6, wherein the controller is further configured to display at least one of the type, amount, and calories of food or drink consumed by the user between the first time and the second time and the amount of exercise, calories burned, and hours of sleep of the user.

8. The electronic device of claim 6, wherein the controller is configured to store at least one of the type, amount, and calories of food or drink in the storage based on information included on a package of the food or drink.

9. The electronic device of claim 6, wherein the controller is configured to store at least one of the type, amount, and calories of food or drink in the storage based on an image of the food or drink.

10. The electronic device of claim 6, wherein the food or drink includes one of health food and medicine.

11. A display method to be executed by an electronic device, the display method comprising:
measuring a contour of an abdomen; and
displaying a first contour and a second contour that are measured at different times in overlap on a display.

12. A display system comprising:
a measurement unit configured to measure a contour of an abdomen; and
a controller configured to display the contour on a display,
the controller being further configured to display a first contour and a second contour that are measured at different times in overlap on the display.
